(19) United States
(12) Patent Application Publication
(10) Pub. No.: US 2018/0002371 A1
(43) Pub. Date: Jan. 4, 2018

(54) BUFALIN PHOSPHATE PRODRUGS AND METHODS OF USE THEREOF

(71) Applicant: BAYLOR COLLEGE OF MEDICINE, Houston, TX (US)

(72) Inventors: Jin WANG, Sugar Land, TX (US); Jianming XU, Bellaire, TX (US); David Michael LONARD, Pearland, TX (US); Bert W. O'MALLEY, Houston, TX (US); Timothy Gerald PALZKILL, Houston, TX (US)

(73) Assignee: Baylor College of Medicine, Houston, TX (US)

(21) Appl. No.: 15/540,407

(22) PCT Filed: Dec. 29, 2015
(86) PCT No.: PCT/US2015/067771

§ 371 Date: Jun. 28, 2017

Related U.S. Application Data
(60) Provisional application No. 62/098,045, filed on Dec. 30, 2014.

(51) Int. Cl. C07J 51/00; A61K 31/5377; G01N 33/574; A61K 31/665; C12Q 1/68

(52) U.S. Cl. CPC C07J 51/00; A61K 31/665; C12Q 1/6886; G01N 33/57415; A61K 31/5377; C12Q 2600/158; G01N 2800/52; C12Q 2600/118

(57) ABSTRACT

Bufalin phosphate prodrugs are provided herein, as well as methods for their use as small molecule inhibitors of steroid receptor coactivator (SRC) family proteins. Methods for using bufalin phosphate prodrugs in treating or preventing cancer are also provided herein.

### CROSS REFERENCE TO PRIORITY APPLICATION

This application claims priority to U.S. Provisional Application No. 62/098,045, filed Dec. 30, 2014, which is incorporated herein by reference in its entirety.

### STATEMENT REGARDING FEDERALLY FUNDED RESEARCH

This invention was made with government support under Grant No. DOD BC120894, awarded by the Department of Defense, and Grant Nos. DK059820 and HD076596, awarded by the National Institutes of Health. The government has certain rights in the invention.

### BACKGROUND

A significant number of transcription factors partner with coactivators that recruit chromatin remodeling factors and interact with the basal transcription machinery. Coactivators have been implicated in cancer cell proliferation, invasion, and metastasis, including the p160 steroid receptor coactivator (SRC) family composed of SRC-1 (NCOA1), SRC-2 (TIF2/GRIP1/NCOA2), and SRC-3 (AIB1/ACTR/NCOA3). Given their broad involvement in many cancers, these coactivators represent candidate molecular targets for new chemotherapeutics.

### SUMMARY

Described herein are steroid receptor coactivator (SRC) inhibitors. Also described herein are methods for their use in treating and/or preventing cancer. The methods include administering to a subject a compound as described herein.

Steroid receptor coactivator inhibitors described herein include compounds of the following formula:

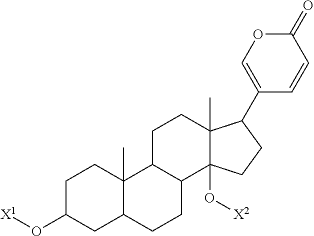

or a pharmaceutically acceptable salt thereof, wherein X¹ and X² are each independently selected from the group consisting of hydrogen and

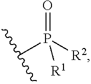

wherein R¹ and R² are each independently selected from the group consisting of hydroxy, substituted or unsubstituted alkoxy, substituted or unsubstituted alkenyloxy, substituted or unsubstituted alkynyloxy, substituted or unsubstituted heteroalkyloxy, substituted or unsubstituted heteroalkenyloxy, substituted or unsubstituted heteroalkynyloxy, substituted or unsubstituted aryloxy, substituted or unsubstituted heteroaryloxy, substituted or unsubstituted cycloalkyloxy, substituted or unsubstituted heterocycloalkyloxy, and substituted or unsubstituted amino; and wherein X¹ and X² are not simultaneously hydrogen.

Optionally, the compound has the following formula:

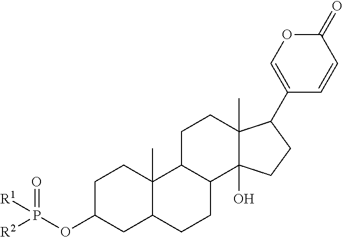

or a pharmaceutically acceptable salt thereof.

Optionally, the compound has the following formula:

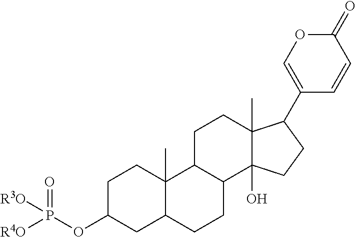

or a pharmaceutically acceptable salt thereof, wherein R³ and R⁴ are each independently selected from the group consisting of hydrogen, substituted or unsubstituted alkyl, substituted or unsubstituted alkenyl, substituted or unsubstituted alkynyl, substituted or unsubstituted heteroalkyl, substituted or unsubstituted heteroalkenyl, substituted or unsubstituted heteroalkynyl, substituted or unsubstituted aryl, substituted or unsubstituted heteroaryl, substituted or unsubstituted cycloalkyl, and substituted or unsubstituted heterocycloalkyl.

Optionally, the compound has the following formula:

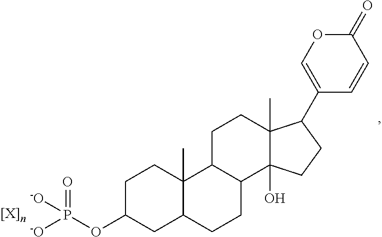

or a pharmaceutically acceptable salt thereof, wherein X is a cation and n is 1 or 2. Optionally, the cation is a metal cation (e.g., an alkali metal cation or an alkaline earth metal cation). Optionally, X is selected from the group consisting of Na⁺, K⁺, Li⁺, and NH₄⁺.

Optionally, the compound has the following formula:

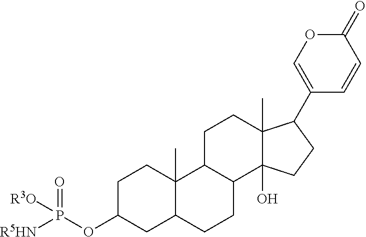

or a pharmaceutically acceptable salt thereof, wherein R³ and R⁵ are each independently selected from the group consisting of hydrogen, substituted or unsubstituted alkyl, substituted or unsubstituted alkenyl, substituted or unsubstituted alkynyl, substituted or unsubstituted heteroalkyl, substituted or unsubstituted heteroalkenyl, substituted or unsubstituted heteroalkynyl, substituted or unsubstituted aryl, substituted or unsubstituted heteroaryl, substituted or unsubstituted cycloalkyl, and substituted or unsubstituted heterocycloalkyl.

Optionally, the compound is

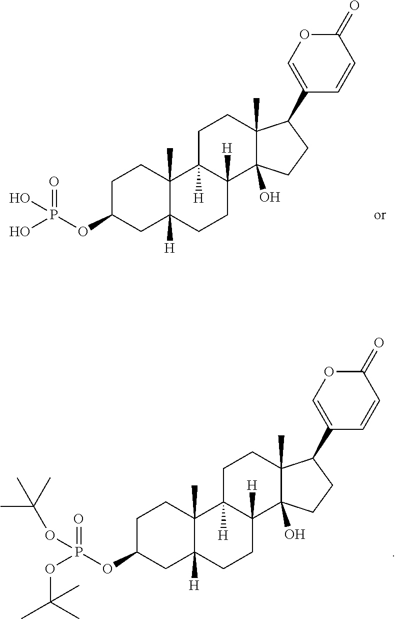

Also described herein is a composition including a compound as described herein and a pharmaceutically acceptable carrier.

Further described herein is a kit including a compound or composition as described herein.

Methods of treating or preventing a steroid receptor coactivator-related disease in a subject are also provided herein. A method of treating or preventing a steroid receptor coactivator-related disease in a subject includes administering to the subject an effective amount of a compound as described herein. Optionally, the steroid receptor coactivator-related disease is cancer. Optionally, the cancer is a poor prognosis or invasive cancer. Optionally, the cancer is breast cancer (e.g., triple negative breast cancer). Optionally, the cancer is pancreatic cancer. Optionally, the cancer is glioblastoma. The glioblastoma is optionally a glioblastoma multiforme tumor (e.g., a pediatric glioblastoma multiforme tumor). Optionally, the cancer is liver cancer, lung cancer, pancreatic cancer, or prostate cancer. Optionally, the steroid receptor coactivator is SRC-3. The methods can further include administering a second compound or composition. Optionally, the second compound or composition is a chemotherapeutic agent (e.g., gefitinib).

Methods of inhibiting a steroid receptor coactivator protein in a cell are also provided herein. A method of inhibiting a steroid receptor coactivator protein in a cell includes contacting a cell with an effective amount of a compound as described herein. The steroid receptor coactivator protein can optionally be SRC-1, SRC-2, or SRC-3. Optionally, the contacting is performed in vitro or in vivo.

Also provided herein are methods of identifying a subject at risk for developing a poor prognosis cancer. A method of identifying a subject at risk for developing a poor prognosis cancer includes obtaining a biological sample from a subject and detecting the expression of SRC-3 in the subject, wherein an increase in expression of SRC-3 in the subject as compared to SRC-3 in a control subject is indicative of a subject at risk for developing a poor prognosis cancer. Optionally, the poor prognosis cancer is triple negative breast cancer.

Further provided herein are methods of treating a subject at risk for developing a poor prognosis cancer. A method of treating a subject at risk for developing a poor prognosis cancer includes identifying a subject at risk for developing a poor prognosis cancer according to the methods described herein and administering to the subject an effective amount of a compound as described herein.

The details of one or more embodiments are set forth in the drawings and the description below. Other features, objects, and advantages will be apparent from the description and drawings, and from the claims.

### DESCRIPTION OF DRAWINGS

FIG. 1 shows an Kaplan-Meier analysis on SRC-3 level and survival rate of TNBC patients. Panel (A) shows overall survival in low SRC-3 expression group and high SRC-3 expression group. Panel (B) shows overall survival in systemically untreated TNBC patients with low or high SRC-3 expressoin. Panel (C) shows distant metastasis free survival in low or high SRC-3 expression group. (D) shows post progression survival in low or high SRC-3 expression group.

FIG. 2 shows that bufalin downregulates SRC-3 and decreases cell viability in TNBC cells. Panel (A) contains a Western blot that shows that bufalin (100 nM) downregulates SRC-3 protein levels in TNBC cells. IC₅₀ values, TNBC subtypes and gene ontologies are also listed. Panel (B) contains a Western blot that shows a dose-dependent downregulation of SRC-3 by bufalin in LM3-3 cells. Panels (C-H) show that bufalin decrease cell viability of TNBC cells including HCC1143 (Panel C), SUM149PT (Panel D), SUM159PT (Panel E), MDA-MB-231 (Panel F) and MDA-MB-231-LM3-3 (Panel G), but not in primary mouse hepatocytes (H). Data are presented as mean±SD.

FIG. 3 shows that bufalin downregulates SRC-3 and reduces cell motility in MDA-MB-231-LM3-3. Panel (A) contains a cell motility assay of LM3-3 cells performed using a Cellomics cell motility kit. The bright areas of 50 images for each sample were analyzed using Image J. Panel (B) shows the treatment of LM3-3 cells with different concentrations of bufalin for 12 h showed minimal toxicity but significant motility reduction. Data are presented as mean±SD.

FIG. 4 shows the synergistic effect of bufalin (0 nM, 5 nM, or 10 nM) and Gefitinib (from left to right for each concentration of bufalin: 0 μM, 5 μM, 10 μM, 15 μM, and 20 μM) in TNBC cells. When combined with 5 or 10 nM of bufalin, cotreatment with the EGFR inhibitor gefitinib synergistically reduced LM3-3 cell viability. Data are presented as mean±SD.

FIG. 5 is a scheme depicting the synthesis of Compound 1 (i.e., 3-phospho-bufalin).

FIG. 6 depicts H-E staining of a bufalin or 3-phospho-bufalin treated mouse heart tissues 24 hours after drug administration. Neither of bufalin or 3-phospho-bufalin caused severe damage to the heart muscle, indicating that the acute toxicity of bufalin is reversible.

FIG. 7 depicts the pharmacokinetics (PK) of bufalin and 3-Phospho-bufalin (p-Buf). Panel (A) is a PK trace of bufalin in mice treated with bufalin (0.5 mg/kg) through i.v. route. Panel (B) is a PK trace of p-buf (orange) and free bufalin generated from p-buf (blue) in mice treated with p-buf (0.5 mg/kg) through i.p. route. The blue and orange dotted lines represent the lower limit of quantification (LLOQ) for bufalin and p-buf in this assay, respectively.

FIG. 8 shows the therapeutic efficacy of 3-phospho-bufalin in an orthotopic TNBC model. Panel (A) shows results from LM3-3 cells inoculated into the mammary fat pads of nude mice (female, 4-5 weeks). The treatment was started 14 days after tumor inoculation. The treatment group was treated with 3-phospho-bufalin (0.75 mg/kg) 3 times per week for 3 weeks (n=6). The control group was treated with PBS (n=6). Tumor volumes were measured three times per week. As shown, 3-phospho-bufalin can significant inhibit TNBC tumor growth. Data are presented as mean±SEM. *, P<0.05; **, P<0.01, by t-test. Panel (B) shows representative images of harvested tumors. Panel (C) provides a comparison of the tumor weights from both the 3-phospho-bufalin treated group and the PBS treated control group. Data are presented in a box plot with mean, minimum and maximum values. P=0.0156 by t-test.

### DETAILED DESCRIPTION

Described herein are steroid receptor coactivator (SRC) inhibitors and methods for their use. In some examples, the compounds are inhibitors for SRC-3 and SRC-1. Optionally, the steroid receptor coactivator inhibitors are phosphate and phosphonate prodrugs of the glycoside bufalin. These prodrugs are water soluble and can be hydrolyzed by endogenous phosphatases under physiological conditions to generate bufalin. The compounds described herein are advantageous as the compounds avoid sudden exposure to high concentrations of free bufalin, which may cause acute cardiotoxicity. Optionally, the bufalin derivatives described herein can be formulated for the treatment of breast cancer and also as broad-spectrum small-molecule inhibitors for cancer.

I. Compounds

A class of SRC inhibitors described herein is represented by Formula I:

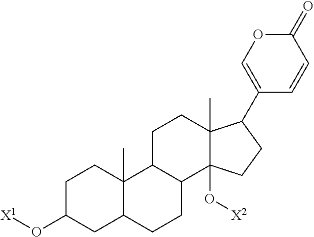

and pharmaceutically acceptable salts or prodrugs thereof.

In Formula I, X¹ and X² are each independently selected from the group consisting of hydrogen or

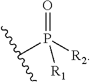

In Formula I, X¹ and X² are not simultaneously hydrogen.

Also in Formula I, R¹ and R² are each independently selected from the group consisting of hydroxy, substituted or unsubstituted alkoxy, substituted or unsubstituted alkenyloxy, substituted or unsubstituted alkynyloxy, substituted or unsubstituted heteroalkyloxy, substituted or unsubstituted heteroalkenyloxy, substituted or unsubstituted heteroalkynyloxy, substituted or unsubstituted aryloxy, substituted or unsubstituted heteroaryloxy, substituted or unsubstituted cycloalkyloxy, substituted or unsubstituted heterocycloalkyloxy, and substituted or unsubstituted amino.

A class of SRC inhibitors described herein is represented by Formula II:

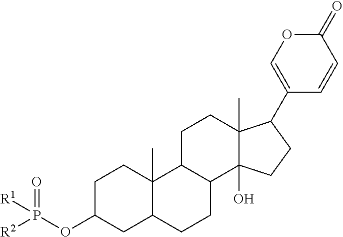

and pharmaceutically acceptable salts or prodrugs thereof.

In Formula II, R¹ and R² are each independently selected from the group consisting of hydroxy, substituted or unsubstituted alkoxy, substituted or unsubstituted alkenyloxy, substituted or unsubstituted alkynyloxy, substituted or unsubstituted heteroalkyloxy, substituted or unsubstituted heteroalkenyloxy, substituted or unsubstituted heteroalkynyloxy, substituted or unsubstituted aryloxy, substituted or unsubstituted heteroaryloxy, substituted or unsubstituted cycloalkyloxy, substituted or unsubstituted heterocycloalkyloxy, and substituted or unsubstituted amino.

In some examples, Formula II is represented by Structure II-A:

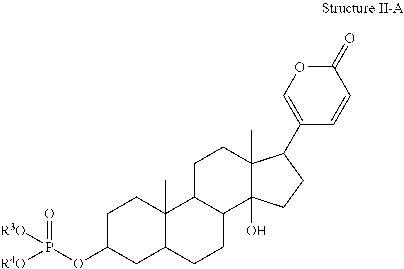

In Structure II-A, R³ and R⁴ are each independently selected from the group consisting of hydrogen, substituted or unsubstituted alkyl, substituted or unsubstituted alkenyl, substituted or unsubstituted alkynyl, substituted or unsubstituted heteroalkyl, substituted or unsubstituted heteroalkenyl, substituted or unsubstituted heteroalkynyl, substituted or unsubstituted aryl, substituted or unsubstituted heteroaryl, substituted or unsubstituted cycloalkyl, and substituted or unsubstituted heterocycloalkyl.

In some examples, Formula II is a salt as represented by Structure II-B:

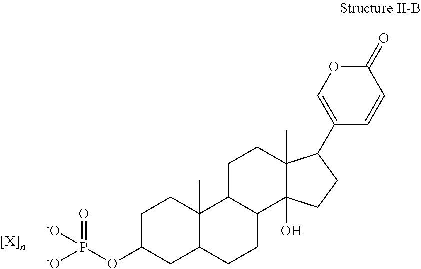

In Structure II-B, X is a cation. Optionally, the cation is a metal cation (e.g., an alkali metal cation or an alkaline earth metal cation) or an ammonium ion. Optionally, X is selected from the group consisting of a sodium cation (Na⁺), a potassium cation (K⁺), a lithium cation (Li⁺), and an ammonium cation (NH₄⁺).

Also in Structure II-B, n is 1 or 2.

In some examples, Formula II is represented by Structure II-C:

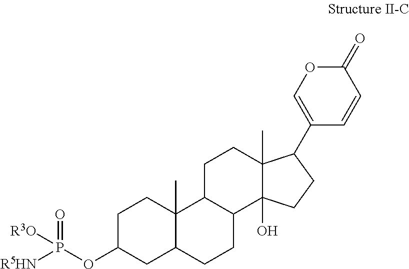

In Structure II-C, R³ is as defined above for Structure II-A.

Also in Structure II-C, R⁵ is selected from the group consisting of hydrogen, substituted or unsubstituted alkyl, substituted or unsubstituted alkenyl, substituted or unsubstituted alkynyl, substituted or unsubstituted heteroalkyl, substituted or unsubstituted heteroalkenyl, substituted or unsubstituted heteroalkynyl, substituted or unsubstituted aryl, substituted or unsubstituted heteroaryl, substituted or unsubstituted cycloalkyl, and substituted or unsubstituted heterocycloalkyl.

Examples of Formula II include Compound 1 and Compound 2 as shown below:

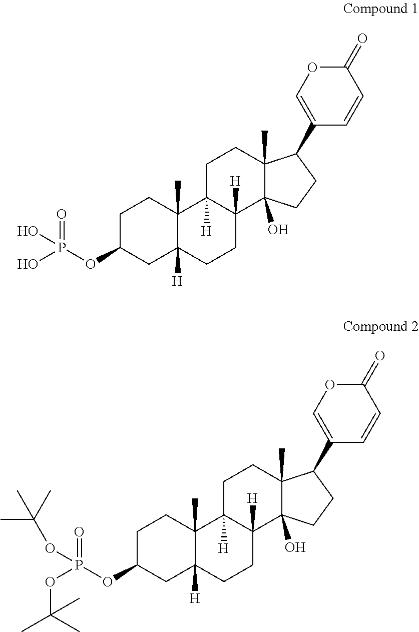

Compound 1 is also referred to herein as 3-Phospho-bufalin or p-Buf.

A class of SRC inhibitors described herein is represented by Formula III:

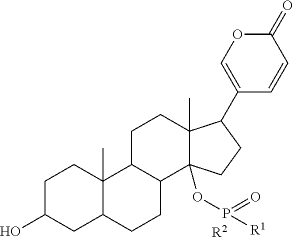

and pharmaceutically acceptable salts or prodrugs thereof.

In Formula III, R¹ and R² are each independently selected from the group consisting of hydroxy, substituted or unsubstituted alkoxy, substituted or unsubstituted alkenyloxy, substituted or unsubstituted alkynyloxy, substituted or unsubstituted heteroalkyloxy, substituted or unsubstituted heteroalkenyloxy, substituted or unsubstituted heteroalkynyloxy, substituted or unsubstituted aryloxy, substituted or unsubstituted heteroaryloxy, substituted or unsubstituted cycloalkyloxy, substituted or unsubstituted heterocycloalkyloxy, and substituted or unsubstituted amino.

In some examples, Formula III is represented by Structure III-A:

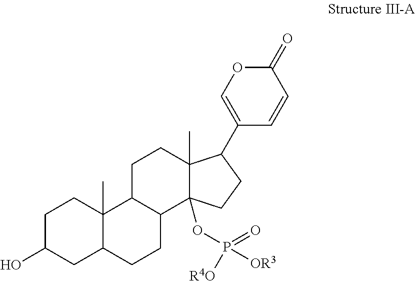

In Structure III-A, R³ and R⁴ are each independently selected from the group consisting of hydrogen, substituted or unsubstituted alkyl, substituted or unsubstituted alkenyl, substituted or unsubstituted alkynyl, substituted or unsubstituted heteroalkyl, substituted or unsubstituted heteroalkenyl, substituted or unsubstituted heteroalkynyl, substituted or unsubstituted aryl, substituted or unsubstituted heteroaryl, substituted or unsubstituted cycloalkyl, and substituted or unsubstituted heterocycloalkyl.

In some examples, Formula III is represented by Structure III-B:

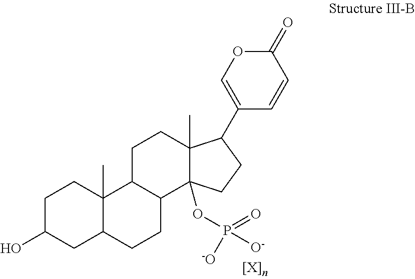

In Structure III-B, X is a cation. Optionally, the cation is a metal cation (e.g., an alkali metal cation or an alkaline earth metal cation) or an ammonium ion. Optionally, X is selected from the group consisting of a sodium cation (Na⁺), a potassium cation (K⁺), a lithium cation (Li⁺), and an ammonium cation (NH₄⁺).

Also in Structure III-B, n is 1 or 2

In some examples, Formula III is represented by Structure III-C:

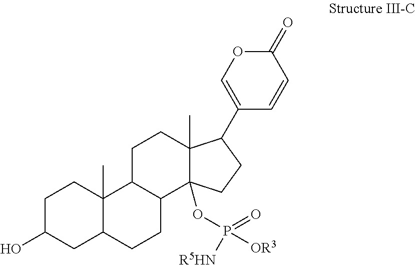

In Structure III-C, R³ is as defined above for Structure III-A.

Also in Structure III-C, R⁵ is selected from the group consisting of hydrogen, substituted or unsubstituted alkyl, substituted or unsubstituted alkenyl, substituted or unsubstituted alkynyl, substituted or unsubstituted heteroalkyl, substituted or unsubstituted heteroalkenyl, substituted or unsubstituted heteroalkynyl, substituted or unsubstituted aryl, substituted or unsubstituted heteroaryl, substituted or unsubstituted cycloalkyl, and substituted or unsubstituted heterocycloalkyl.

Examples of Formula III include Compound 3 and Compound 4 as shown below:

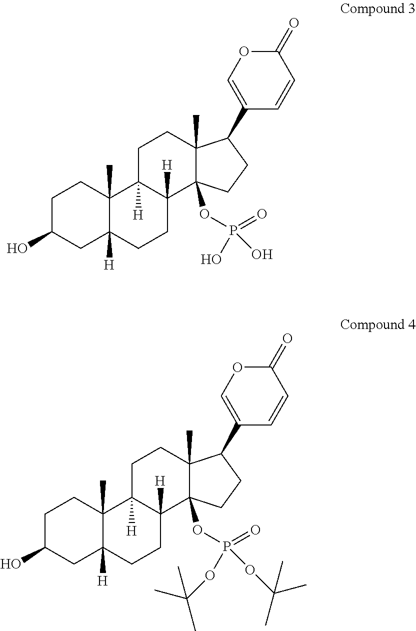

As used herein, the terms alkyl, alkenyl, and alkynyl include straight- and branched-chain monovalent substituents. Examples include methyl, ethyl, isobutyl, 3-butynyl, and the like. Ranges of these groups useful with the compounds and methods described herein include C₁-C₂₀ alkyl, C₂-C₂₀ alkenyl, and C₂-C₂₀ alkynyl. Additional ranges of these groups useful with the compounds and methods described herein include C₁-C₁₂ alkyl, C₂-C₁₂ alkenyl, C₂-C₁₂ alkynyl, C₁-C₆ alkyl, C₂-C₆ alkenyl, C₂-C₆ alkynyl, C₁-C₄ alkyl, C₂-C₄ alkenyl, and C₂-C₄ alkynyl.

Heteroalkyl, heteroalkenyl, and heteroalkynyl are defined similarly as alkyl, alkenyl, and alkynyl, but can contain O, S, or N heteroatoms or combinations thereof within the backbone. Ranges of these groups useful with the compounds and methods described herein include C₁-C₂₀ heteroalkyl, C₂-C₂₀ heteroalkenyl, and C₂-C₂₀ heteroalkynyl. Additional ranges of these groups useful with the compounds and methods described herein include C₁-C₁₂ heteroalkyl, C₂-C₁₂ heteroalkenyl, C₂-C₁₂ heteroalkynyl, C₁-C₆ heteroalkyl, C₂-C₆ heteroalkenyl, C₂-C₆ heteroalkynyl, C₁-C₄ heteroalkyl, C₂-C₄ heteroalkenyl, and C₂-C₄ heteroalkynyl.

The terms cycloalkyl, cycloalkenyl, and cycloalkynyl include cyclic alkyl groups having a single cyclic ring or multiple condensed rings. Examples include cyclohexyl, cyclopentylethyl, and adamantanyl. Ranges of these groups useful with the compounds and methods described herein include C₃-C₂₀ cycloalkyl, C₃-C₂₀ cycloalkenyl, and C₃-C₂₀ cycloalkynyl. Additional ranges of these groups useful with the compounds and methods described herein include C₅-C₁₂ cycloalkyl, C₅-C₁₂ cycloalkenyl, C₅-C₁₂ cycloalkynyl, C₅-C₆ cycloalkyl, C₅-C₆ cycloalkenyl, and C₅-C₆ cycloalkynyl.

The terms heterocycloalkyl, heterocycloalkenyl, and heterocycloalkynyl are defined similarly as cycloalkyl, cycloalkenyl, and cycloalkynyl, but can contain O, S, or N heteroatoms or combinations thereof within the cyclic backbone. Ranges of these groups useful with the compounds and methods described herein include C₃-C₂₀ heterocycloalkyl, C₃-C₂₀ heterocycloalkenyl, and C₃-C₂₀ heterocycloalkynyl. Additional ranges of these groups useful with the compounds and methods described herein include C₅-C₁₂ heterocycloalkyl, C₅-C₁₂ heterocycloalkenyl, C₅-C₁₂ heterocycloalkynyl, C₅-C₆ heterocycloalkyl, C₅-C₆ heterocycloalkenyl, and C₅-C₆ heterocycloalkynyl.

Aryl molecules include, for example, cyclic hydrocarbons that incorporate one or more planar sets of, typically, six carbon atoms that are connected by delocalized electrons numbering the same as if they consisted of alternating single and double covalent bonds. An example of an aryl molecule is benzene. Heteroaryl molecules include substitutions along their main cyclic chain of atoms such as O, N, or S. When heteroatoms are introduced, a set of five atoms, e.g., four carbon and a heteroatom, can create an aromatic system. Examples of heteroaryl molecules include furan, pyrrole, thiophene, imadazole, oxazole, pyridine, and pyrazine. Aryl and heteroaryl molecules can also include additional fused rings, for example, benzofuran, indole, benzothiophene, naphthalene, anthracene, and quinoline. The aryl and heteroaryl molecules can be attached at any position on the ring, unless otherwise noted.

The term alkoxy as used herein is an alkyl group bound through a single, terminal ether linkage. The term aryloxy as used herein is an aryl group bound through a single, terminal ether linkage. Likewise, the terms alkenyloxy, alkynyloxy, heteroalkyloxy, heteroalkenyloxy, heteroalkynyloxy, heteroaryloxy, cycloalkyloxy, and heterocycloalkyloxy as used herein are an alkenyloxy, alkynyloxy, heteroalkyloxy, heteroalkenyloxy, heteroalkynyloxy, heteroaryloxy, cycloalkyloxy, and heterocycloalkyloxy group, respectively, bound through a single, terminal ether linkage.

The term hydroxy as used herein is represented by the formula —OH.

The terms amine or amino as used herein are represented by the formula —NZ¹Z², where Z¹ and Z² can each be substitution group as described herein, such as hydrogen, an alkyl, halogenated alkyl, alkenyl, alkynyl, aryl, heteroaryl, cycloalkyl, cycloalkenyl, heterocycloalkyl, or heterocycloalkenyl group described above.

The alkoxy, aryloxy, amino, alkyl, alkenyl, alkynyl, aryl, heteroalkyl, heteroalkenyl, heteroalkynyl, heteroaryl, cycloalkyl, or heterocycloalkyl molecules used herein can be substituted or unsubstituted. As used herein, the term substituted includes the addition of an alkoxy, aryloxy, amino, alkyl, alkenyl, alkynyl, aryl, heteroalkyl, heteroalkenyl, heteroalkynyl, heteroaryl, cycloalkyl, or heterocycloalkyl group to a position attached to the main chain of the alkoxy, aryloxy, amino, alkyl, alkenyl, alkynyl, aryl, heteroalkyl, heteroalkenyl, heteroalkynyl, heteroaryl, cycloalkyl, or heterocycloalkyl, e.g., the replacement of a hydrogen by one of these molecules. Examples of substitution groups include, but are not limited to, hydroxy, halogen (e.g., F, Br, Cl, or I), and carboxyl groups. Conversely, as used herein, the term unsubstituted indicates the alkoxy, aryloxy, amino, alkyl, alkenyl, alkynyl, aryl, heteroalkyl, heteroalkenyl, heteroalkynyl, heteroaryl, cycloalkyl, or heterocycloalkyl has a full complement of hydrogens, i.e., commensurate with its saturation level, with no substitutions, e.g., linear decane (—(CH₂)₉—CH₃).

II. Methods of Making the Compounds

The compounds described herein can be prepared in a variety of ways. The compounds can be synthesized using various synthetic methods. At least some of these methods are known in the art of synthetic organic chemistry. The compounds described herein can be prepared from readily available starting materials. Optimum reaction conditions can vary with the particular reactants or solvent used, but such conditions can be determined by one skilled in the art by routine optimization procedures.

Variations on Formula I, Formula II, and Formula III include the addition, subtraction, or movement of the various constituents as described for each compound. Similarly, when one or more chiral centers are present in a molecule, all possible chiral variants are included. Additionally, compound synthesis can involve the protection and deprotection of various chemical groups. The use of protection and deprotection, and the selection of appropriate protecting groups can be determined by one skilled in the art. The chemistry of protecting groups can be found, for example, in Greene, et al., Protective Groups in Organic Synthesis, 2d. Ed., Wiley & Sons, 1991, which is incorporated herein by reference in its entirety.

Reactions to produce the compounds described herein can be carried out in solvents, which can be selected by one of skill in the art of organic synthesis. Solvents can be substantially nonreactive with the starting materials (reactants), the intermediates, or products under the conditions at which the reactions are carried out, i.e., temperature and pressure. Reactions can be carried out in one solvent or a mixture of more than one solvent. Product or intermediate formation can be monitored according to any suitable method known in the art. For example, product formation can be monitored by spectroscopic means, such as nuclear magnetic resonance spectroscopy (e.g., ¹H or ¹³C) infrared spectroscopy, spectrophotometry (e.g., UV-visible), or mass spectrometry, or by chromatography such as high performance liquid chromatography (HPLC) or thin layer chromatography.

Exemplary methods for synthesizing compounds as described herein are provided in Example 1 below.

III. Pharmaceutical Formulations

The compounds described herein or derivatives thereof can be provided in a pharmaceutical composition. Depending on the intended mode of administration, the pharmaceutical composition can be in the form of solid, semi-solid or liquid dosage forms, such as, for example, tablets, suppositories, pills, capsules, powders, liquids, or suspensions, preferably in unit dosage form suitable for single administration of a precise dosage. The compositions will include a therapeutically effective amount of the compound described herein or derivatives thereof in combination with a pharmaceutically acceptable carrier and, in addition, may include other medicinal agents, pharmaceutical agents, carriers, or diluents. By pharmaceutically acceptable is meant a material that is not biologically or otherwise undesirable, which can be administered to an individual along with the selected compound without causing unacceptable biological effects or interacting in a deleterious manner with the other components of the pharmaceutical composition in which it is contained.

As used herein, the term carrier encompasses any excipient, diluent, filler, salt, buffer, stabilizer, solubilizer, lipid, stabilizer, or other material well known in the art for use in pharmaceutical formulations. The choice of a carrier for use in a composition will depend upon the intended route of administration for the composition. The preparation of pharmaceutically acceptable carriers and formulations containing these materials is described in, e.g., Remington's Pharmaceutical Sciences, 21st Edition, ed. University of the Sciences in Philadelphia, Lippincott, Williams & Wilkins, Philadelphia Pa., 2005. Examples of physiologically acceptable carriers include buffers, such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers, such as polyvinylpyrrolidone; amino acids such as glycine, glutamine, asparagine, arginine or lysine; monosaccharides, disaccharides, and other carbohydrates, including glucose, mannose, or dextrins; chelating agents, such as EDTA; sugar alcohols, such as mannitol or sorbitol; salt-forming counterions, such as sodium; and/or nonionic surfactants, such as TWEEN® (ICI, Inc.; Bridgewater, N.J.), polyethylene glycol (PEG), and PLURONICSTM (BASF; Florham Park, N.J.).

Compositions containing the compound described herein or derivatives thereof suitable for parenteral injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents or vehicles include water, ethanol, polyols (propyleneglycol, polyethyleneglycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions and by the use of surfactants.

These compositions may also contain adjuvants, such as preserving, wetting, emulsifying, and dispensing agents. Prevention of the action of microorganisms can be promoted by various antibacterial and antifungal agents, for example, parabens, chlorobutanol, phenol, sorbic acid, and the like. Isotonic agents, for example, sugars, sodium chloride, and the like may also be included. Prolonged absorption of the injectable pharmaceutical form can be brought about by the use of agents delaying absorption, for example, aluminum monostearate and gelatin.

Solid dosage forms for oral administration of the compounds described herein or derivatives thereof include capsules, tablets, pills, powders, and granules. In such solid dosage forms, the compounds described herein or derivatives thereof is admixed with at least one inert customary excipient (or carrier), such as sodium citrate or dicalcium phosphate, or (a) fillers or extenders, as for example, starches, lactose, sucrose, glucose, mannitol, and silicic acid, (b) binders, as for example, carboxymethylcellulose, alignates, gelatin, polyvinylpyrrolidone, sucrose, and acacia, (c) humectants, as for example, glycerol, (d) disintegrating agents, as for example, agar-agar, calcium carbonate, potato or tapioca starch, alginic acid, certain complex silicates, and sodium carbonate, (e) solution retarders, as for example, paraffin, (f) absorption accelerators, as for example, quaternary ammonium compounds, (g) wetting agents, as for example, cetyl alcohol, and glycerol monostearate, (h) adsorbents, as for example, kaolin and bentonite, and (i) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents.

Solid compositions of a similar type may also be employed as fillers in soft and hard-filled gelatin capsules using such excipients as lactose or milk sugar as well as high molecular weight polyethyleneglycols, and the like.

Solid dosage forms such as tablets, dragees, capsules, pills, and granules can be prepared with coatings and shells, such as enteric coatings and others known in the art. They may contain opacifying agents and can also be of such composition that they release the active compound or compounds in a certain part of the intestinal tract in a delayed manner. Examples of embedding compositions that can be used are polymeric substances and waxes. The active compounds can also be in micro-encapsulated form, if appropriate, with one or more of the above-mentioned excipients.

Liquid dosage forms for oral administration of the compounds described herein or derivatives thereof include pharmaceutically acceptable emulsions, solutions, suspensions, syrups, and elixirs. In addition to the active compounds, the liquid dosage forms may contain inert diluents commonly used in the art, such as water or other solvents, solubilizing agents, and emulsifiers, as for example, ethyl alcohol, isopropyl alcohol, ethyl carbonate, ethyl acetate, benzyl alcohol, benzyl benzoate, propyleneglycol, 1,3-butyleneglycol, dimethylformamide, oils, in particular, cottonseed oil, groundnut oil, corn germ oil, olive oil, castor oil, sesame oil, glycerol, tetrahydrofurfuryl alcohol, polyethyleneglycols, and fatty acid esters of sorbitan, or mixtures of these substances, and the like.

Besides such inert diluents, the composition can also include additional agents, such as wetting, emulsifying, suspending, sweetening, flavoring, or perfuming agents.

Suspensions, in addition to the active compounds, may contain additional agents, as for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like.

Compositions of the compounds described herein or derivatives thereof for rectal administrations are optionally suppositories, which can be prepared by mixing the compounds with suitable non-irritating excipients or carriers, such as cocoa butter, polyethyleneglycol or a suppository wax, which are solid at ordinary temperatures but liquid at body temperature and, therefore, melt in the rectum or vaginal cavity and release the active component.

Dosage forms for topical administration of the compounds described herein or derivatives thereof include ointments, powders, sprays, and inhalants. The compounds described herein or derivatives thereof are admixed under sterile conditions with a physiologically acceptable carrier and any preservatives, buffers, or propellants as may be required. Ophthalmic formulations, ointments, powders, and solutions are also contemplated as being within the scope of the compositions.

The compositions can include one or more of the compounds described herein and a pharmaceutically acceptable carrier. As used herein, the term pharmaceutically acceptable salt refers to those salts of the compound described herein or derivatives thereof that are, within the scope of sound medical judgment, suitable for use in contact with the tissues of subjects without undue toxicity, irritation, allergic response, and the like, commensurate with a reasonable benefit/risk ratio, and effective for their intended use, as well as the zwitterionic forms, where possible, of the compounds described herein. The term salts refers to the relatively non-toxic, inorganic and organic acid addition salts of the compounds described herein. These salts can be prepared in situ during the isolation and purification of the compounds or by separately reacting the purified compound in its free base form with a suitable organic or inorganic acid and isolating the salt thus formed. Representative salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate mesylate, glucoheptonate, lactobionate, methane sulphonate, and laurylsulphonate salts, and the like. These may include cations based on the alkali and alkaline earth metals, such as sodium, lithium, potassium, calcium, magnesium, and the like, as well as non-toxic ammonium, quaternary ammonium, and amine cations including, but not limited to ammonium, tetramethylammonium, tetraethylammonium, methylamine, dimethylamine, trimethylamine, triethylamine, ethylamine, and the like. (See S. M. Barge et al., J. Pharm. Sci. (1977) 66, 1, which is incorporated herein by reference in its entirety, at least, for compositions taught therein.)

Administration of the compounds and compositions described herein or pharmaceutically acceptable salts thereof can be carried out using therapeutically effective amounts of the compounds and compositions described herein or pharmaceutically acceptable salts thereof as described herein for periods of time effective to treat a disorder. The effective amount of the compounds and compositions described herein or pharmaceutically acceptable salts thereof as described herein may be determined by one of ordinary skill in the art and includes exemplary dosage amounts for a mammal of from about 0.5 to about 200 mg/kg of body weight of active compound per day, which may be administered in a single dose or in the form of individual divided doses, such as from 1 to 4 times per day. Alternatively, the dosage amount can be from about 0.5 to about 150 mg/kg of body weight of active compound per day, about 0.5 to 100 mg/kg of body weight of active compound per day, about 0.5 to about 75mg/kg of body weight of active compound per day, about 0.5 to about 50mg/kg of body weight of active compound per day, about 0.01 to about 50mg/kg of body weight of active compound per day, about 0.05 to about 25 mg/kg of body weight of active compound per day, about 0.1 to about 25 mg/kg of body weight of active compound per day, about 0.5 to about 25 mg/kg of body weight of active compound per day, about 1 to about 20 mg/kg of body weight of active compound per day, about 1 to about 10 mg/kg of body weight of active compound per day, about 20mg/kg of body weight of active compound per day, about 10 mg/kg of body weight of active compound per day, about 5 mg/kg of body weight of active compound per day, about 2.5 mg/kg of body weight of active compound per day, about 1.0 mg/kg of body weight of active compound per day, or about 0.5 mg/kg of body weight of active compound per day, or any range derivable therein. Optionally, the dosage amounts are from about 0.01 mg/kg to about 10 mg/kg of body weight of active compound per day. Optionally, the dosage amount is from about 0.01 mg/kg to about 5 mg/kg. Optionally, the dosage amount is from about 0.01 mg/kg to about 2.5 mg/kg.

Those of skill in the art will understand that the specific dose level and frequency of dosage for any particular subject may be varied and will depend upon a variety of factors, including the activity of the specific compound employed, the metabolic stability and length of action of that compound, the species, age, body weight, general health, sex and diet of the subject, the mode and time of administration, rate of excretion, drug combination, and severity of the particular condition.

The precise dose to be employed in the formulation will also depend on the route of administration, and the seriousness of the disease or disorder, and should be decided according to the judgment of the practitioner and each subject's circumstances. Effective doses can be extrapolated from dose-response curves derived from in vitro or animal model test systems. Further, depending on the route of administration, one of skill in the art would know how to determine doses that result in a plasma concentration for a desired level of response in the cells, tissues and/or organs of a subject.

IV. Methods of Use

Provided herein are methods to treat, prevent, or ameliorate a steroid receptor coactivator-related disease in a subject. The methods include administering to a subject an effective amount of one or more of the compounds or compositions described herein, or a pharmaceutically acceptable salt or prodrug thereof. Effective amount, when used to describe an amount of compound in a method, refers to the amount of a compound that achieves the desired pharmacological effect or other biological effect. The effective amount can be, for example, the concentrations of compounds at which SRC is inhibited in vitro, as provided herein. Also contemplated is a method that includes administering to the subject an amount of one or more compounds described herein such that an in vivo concentration at a target cell in the subject corresponding to the concentration administered in vitro is achieved.

The compounds and compositions described herein or pharmaceutically acceptable salts thereof are useful for treating steroid receptor coactivator-related diseases in humans, including, without limitation, pediatric and geriatric populations, and in animals, e.g., veterinary applications.

Optionally, the steroid receptor coactivator-related disease is an SRC-1 related disease. Optionally, the steroid receptor coactivator-related disease is an SRC-2 related disease. Optionally, the steroid receptor coactivator-related disease is an SRC-3 related disease.

In some embodiments, the steroid receptor coactivator-related disease is cancer. Optionally, the cancer is a poor prognosis cancer. The term poor prognosis, as used herein, refers to a prospect of recovery from a disease, infection, or medical condition that is associated with a diminished likelihood of a positive outcome. In relation to a disease such as cancer, a poor prognosis may be associated with a reduced patient survival rate, reduced patient survival time, higher likelihood of metastatic progression of said cancer cells, and/or higher likelihood of chemoresistance of said cancer cells. Optionally, a poor prognosis cancer can be a cancer associated with a patient survival rate of 50% or less. Optionally, a poor prognosis cancer can be a cancer associated with a patient survival time of five years or less after diagnosis. In some embodiments, the cancer is an invasive cancer.

Optionally, the cancer is a cancer that has an increased expression of SRC-1, SRC-2, and/or SRC-3 as compared to non-cancerous cells of the same cell type. Optionally, the cancer is bladder cancer, brain cancer, breast cancer, colorectal cancer (e.g., colon cancer, rectal cancer), cervical cancer, chondrosarcoma, endometrial cancer, gastrointestinal cancer, gastric cancer, genitourinary cancer, head and neck cancer, hepatocellular carcinoma, liver cancer, lung cancer, ovarian cancer, pancreatic cancer, prostate cancer, renal cancer, skin cancer, or testicular cancer. Optionally, the breast cancer is triple negative breast cancer. As used herein, triple negative breast cancer (TNBC) refers to a subtype of breast cancer that lacks detectable protein expression of the estrogen receptor (ER) and progesterone receptor (PR) and the absence of HER2 protein over expression. In other words, TNBC refers to an immunophenotype of breast cancer that is immunologically negative to ER, PR, and HER2.

Optionally, the cancer is glioblastoma. In some examples, the glioblastoma is a glioblastoma multiforme tumor. Optionally, the glioblastoma multiforme tumor is a pediatric glioblastoma multiforme tumor. The methods of treating glioblastoma include administering to the subject a compound as described herein. Optionally, the methods of treating glioblastoma include methods of suppressing the growth of glioblastoma cells in the subject.

In some embodiments, the steroid receptor coactivator-related disease is obesity. In some embodiments, the steroid receptor coactivator-related disease is human immunodeficiency virus (HIV). Optionally, the HIV is HIV type 1 (HIV-1). Optionally, the HIV is HIV type 2 (HIV-2).

The methods of treating or preventing an SRC-related disease (e.g., cancer) in a subject can further comprise administering to the subject one or more additional agents. The one or more additional agents and the compounds described herein or pharmaceutically acceptable salts or prodrugs thereof can be administered in any order, including concomitant, simultaneous, or sequential administration. Sequential administration can be administration in a temporally spaced order of up to several days apart. The methods can also include more than a single administration of the one or more additional agents and/or the compounds described herein or pharmaceutically acceptable salts or prodrugs thereof. The administration of the one or more additional agents and the compounds described herein or pharmaceutically acceptable salts or prodrugs thereof can be by the same or different routes and concurrently or sequentially.

Additional therapeutic agents include, but are not limited to, chemotherapeutic agents. A chemotherapeutic agent is a compound or composition effective in inhibiting or arresting the growth of an abnormally growing cell. Thus, such an agent may be used therapeutically to treat cancer as well as other diseases marked by abnormal cell growth. Illustrative examples of chemotherapeutic compounds include, but are not limited to, bexarotene, gefitinib, erlotinib, gemcitabine, paclitaxel, docetaxel, topotecan, irinotecan, temozolomide, carmustine, vinorelbine, capecitabine, leucovorin, oxaliplatin, bevacizumab, cetuximab, panitumumab, bortezomib, oblimersen, hexamethylmelamine, ifosfamide, CPT-11, deflunomide, cycloheximide, dicarbazine, asparaginase, mitotant, vinblastine sulfate, carboplatin, colchicine, etoposide, melphalan, 6-mercaptopurine, teniposide, vinblastine, antibiotic derivatives (e.g. anthracyclines such as doxorubicin, liposomal doxorubicin, and diethylstilbestrol doxorubicin, bleomycin, daunorubicin, and dactinomycin); antiestrogens (e.g., tamoxifen); antimetabolites (e.g., fluorouracil (FU), 5-FU, methotrexate, floxuridine, interferon alpha-2B, glutamic acid, plicamycin, mercaptopurine, and 6-thioguanine); cytotoxic agents (e.g., carmustine, BCNU, lomustine, CCNU, cytosine arabinoside, cyclophosphamide, estramustine, hydroxyurea, procarbazine, mitomycin, busulfan, cisplatin, vincristine and vincristine sulfate); hormones (e.g., medroxyprogesterone, estramustine phosphate sodium, ethinyl estradiol, estradiol, megestrol acetate, methyltestosterone, diethylstilbestrol diphosphate, chlorotrianisene, and testolactone); nitrogen mustard derivatives (e.g., mephalen, chlorambucil, mechlorethamine (nitrogen mustard) and thiotepa); and steroids (e.g., bethamethasone sodium phosphate).

Any of the aforementioned therapeutic agents can be used in any combination with the compositions described herein. Combinations are administered either concomitantly (e.g., as an admixture), separately but simultaneously (e.g., via separate intravenous lines into the same subject), or sequentially (e.g., one of the compounds or agents is given first followed by the second). Thus, the term combination is used to refer to concomitant, simultaneous, or sequential administration of two or more agents.

Optionally, a compound or therapeutic agent as described herein may be administered in combination with a radiation therapy, an immunotherapy, a gene therapy, or a surgery.

The methods and compounds as described herein are useful for both prophylactic and therapeutic treatment. For prophylactic use, a therapeutically effective amount of the compounds and compositions or pharmaceutically acceptable salts thereof as described herein are administered to a subject prior to onset (e.g., before obvious signs of a steroid receptor coactivator-related disease), during early onset (e.g., upon initial signs and symptoms of a steroid receptor coactivator-related disease), or after the development of a steroid receptor coactivator-related disease. Prophylactic administration can occur for several days to years prior to the manifestation of symptoms of a steroid receptor coactivator-related disease. Therapeutic treatment involves administering to a subject a therapeutically effective amount of the compounds and compositions or pharmaceutically acceptable salts thereof as described herein after a steroid receptor coactivator-related disease is diagnosed.

The compounds described herein are also useful in inhibiting a steroid receptor coactivator protein in a cell. The methods of inhibiting a steroid receptor coactivator protein in a cell includes contacting a cell with an effective amount of one or more of the compounds as described herein. Optionally, the steroid receptor coactivator protein is one or more of SRC-1, SRC-2, or SRC-3. Optionally, the contacting is performed in vivo. Optionally, the contacting is performed in vitro.

The methods herein for prophylactic and therapeutic treatment optionally comprise selecting a subject with or at risk of developing a steroid receptor coactivator-related disease. A skilled artisan can make such a determination using, for example, a variety of prognostic and diagnostic methods, including, for example, a personal or family history of the disease or condition, clinical tests (e.g., imaging, biopsy, genetic tests), and the like. Optionally, the methods herein can be used for preventing relapse of cancer in a subject in remission (e.g., a subject that previously had cancer).

Also provided herein is a method for identifying a subject at risk for developing a poor prognosis cancer. The method can include the steps of obtaining a biological sample from the subject (e.g., isolating a sample from the subject) and determining the level of expression of a steroid receptor coactivator (e.g., SRC-3) in the sample, wherein an increase in expression as compared to a control indicates the subject has or is at risk for developing cancer. An increased or higher level in expression or activity of a steroid receptor coactivator as compared to a control means that the level of expression or activity of a steroid receptor coactivator is higher in the biological sample from a subject being tested than in a control sample. As used throughout, higher or increase as compared to a control refer to increases above a control. As used herein, control refers to a reference standard from, for example, an untreated sample or subject, from a subject without cancer, an untreated subject with cancer. By way of another example, a control level can be the level of expression or activity in a control sample in the absence of a stimulus. Alternatively, a control level can be the level of expression or activity in a control sample from a subject or group of subjects without cancer. An increased or high level is optionally statistically higher than a selected control using at least one acceptable statistical analysis method.

As used herein a biological sample which is subjected to testing is a sample derived from a subject and includes, but is not limited to, any cell, tissue or biological fluid. The sample can be, but is not limited to, peripheral blood, plasma, urine, saliva, gastric secretion, feces, bone marrow specimens, primary or metastatic tumor biopsy, embedded tissue sections, frozen tissue sections, cell preparations, cytological preparations, exfoliate samples (e.g., sputum), fine needle aspirations, amnion cells, fresh tissue, dry tissue, and cultured cells or tissue. The biological sample can also be whole cells or cell organelles (e.g., nuclei). A biological sample can also include a partially purified sample, cell culture, or a cell line.

Assay techniques that can be used to determine levels of expression in a sample are well-known to those of skill in the art. Such assay methods include radioimmunoassays, reverse transcriptase PCR (RT-PCR) assays, immunohistochemistry assays, in situ hybridization assays, competitive-binding assays, Western blot analyses, ELISA assays and proteomic approaches, two-dimensional gel electrophoresis (2D electrophoresis) and non-gel based approaches such as mass spectrometry or protein interaction profiling. Assays also include, but are not limited to, competitive and non-competitive assay systems using techniques such as radioimmunoassays, enzyme immunoassays (EIA), enzyme linked immunosorbent assay (ELISA), sandwich immunoassays, precipitin reactions, gel diffusion reactions, immunodiffusion assays, agglutination assays, complement-fixation assays, immunoradiometric assays, fluorescent immunoassays, protein A immunoassays, and immunoelectrophoresis assays. For examples of immunoassay methods, see U.S. Pat. No. 4,845,026 and U.S. Pat. No. 5,006,459.

For diagnostic methods, an antigen binding partner, for example, an antibody, can be labeled with a detectable moiety and used to detect the antigen in a sample. The antibody can be directly labeled or indirectly labeled (e.g., by a secondary or tertiary antibody that is labeled with a detectable moiety). Numerous labels are available including, but not limited to radioisotopes, fluorescent labels, and enzyme-substrate labels. Radioisotopes include, for example, 35S, ¹⁴C, ¹²⁵I, ³H, and ¹³¹I. Fluorescent labels include, for example, rare earth chelates (europium chelates), fluorescein and its derivatives, rhodamine and its derivatives, dansyl, Lissamine, phycoerythrin and Texas Red. The labels can be conjugated to the antigen binding partner using the techniques disclosed in Current Protocols in Immunology, Volumes 1 and 2, Coligen et al., Ed., Wiley-Interscience, New York, N.Y., Pubs., (1991), for example.

When using enzyme-substrate labels, the enzyme preferably catalyses a chemical alteration of the chromogenic substrate which can be measured using various techniques. For example, the enzyme may catalyze a color change in a substrate, which can be measured spectrophotometrically. Alternatively, the enzyme may alter the fluorescence or chemiluminescence of the substrate. The chemiluminescent substrate becomes electronically excited by a chemical reaction and may then emit light which can be measured (using a chemiluminometer, for example) or donates energy to a fluorescent acceptor. Examples of enzymatic labels include luciferases (e.g., firefly luciferase and bacterial luciferase), luciferin, 2,3-dihydrophthalazinediones, malate dehydrogenase, urease, peroxidase such as horseradish peroxidase (HRPO), alkaline phosphatase, β-galactosidase, glucoamylase, lysozyme, saccharide oxidases (e.g., glucose oxidase, galactose oxidase, and glucose-6-phosphate dehydrogenase), heterocyclic oxidases (such as uricase and xanthine oxidase), lactoperoxidase, microperoxidase, and the like. Techniques for conjugating enzymes are described in O'Sullivan et al., Methods for the Preparation of Enzyme-Antibody Conjugates for use in Enzyme Immunoassay, in Methods in Enzym. (ed J. Langone & H. Van Vunakis), Academic press, New York, 73: 147-166 (1981). Examples of enzyme-substrate combinations include, for example, horseradish peroxidase (HRPO) with hydrogen peroxidase as a substrate, alkaline phosphatase (AP) with para-Nitrophenyl phosphate as chromogenic substrate, and β-D-galactosidase (β-D-Gal) with a chromogenic substrate (e.g. p-nitrophenyl-β-D-galactosidase) or fluorogenic substrate 4-methylumbelliferyl-β-D-galactosidase.

In an ELISA assay, an antibody is prepared, if not readily available from a commercial source, specific to an antigen. In addition, a reporter antibody generally is prepared which binds specifically to the antigen. The reporter antibody is attached to a detectable reagent such as a radioactive, fluorescent or enzymatic reagent, for example horseradish peroxidase enzyme or alkaline phosphatase. To carry out the ELISA, antibody specific to antigen is incubated on a solid support, e.g., a polystyrene dish, that binds the antibody. Any free protein binding sites on the dish are then covered by incubating with a non-specific protein such as bovine serum albumin. Next, the sample to be analyzed is incubated in the dish, during which time the antigen binds to the specific antibody attached to the polystyrene dish. Unbound sample is washed out with buffer. A reporter antibody specifically directed to the antigen and linked to a detectable reagent such as horseradish peroxidase is placed in the dish resulting in binding of the reporter antibody to any antibody bound to the antigen. Unattached reporter antibody is then washed out. Reagents for peroxidase activity, including a colorimetric substrate are then added to the dish. Immobilized peroxidase, linked to antibodies, produces a colored reaction product. The amount of color developed in a given time period is proportional to the amount of antigen present in the sample. Quantitative results typically are obtained by reference to a standard curve.

A competition assay can also be employed wherein antibodies specific to antigen are attached to a solid support and labeled antigen and a sample derived from the subject or control are passed over the solid support. The amount of label detected which is attached to the solid support can be correlated to a quantity of antigen in the sample.

Of the proteomic approaches, 2D electrophoresis is a technique well known to those in the art. Isolation of individual proteins from a sample such as serum is accomplished using sequential separation of proteins by different characteristics usually on polyacrylamide gels. First, proteins are separated by size using an electric current. The current acts uniformly on all proteins, so smaller proteins move farther on the gel than larger proteins. The second dimension applies a current perpendicular to the first and separates proteins not on the basis of size but on the specific electric charge carried by each protein. Since no two proteins with different sequences are identical on the basis of both size and charge, the result of a 2D separation is a square gel in which each protein occupies a unique spot. Analysis of the spots with chemical or antibody probes, or subsequent protein microsequencing can reveal the relative abundance of a given protein and the identity of the proteins in the sample.

Optionally, a genetic sample from the biological sample can be obtained. The genetic sample comprises a nucleic acid, preferably RNA and/or DNA. For example, in determining the expression of genes mRNA can be obtained from the biological sample, and the mRNA may be reverse transcribed into cDNA for further analysis. Alternatively, the mRNA itself is used in determining the expression of genes.

A genetic sample may be obtained from the biological sample using any techniques known in the art (Ausubel et al. Current Protocols in Molecular Biology (John Wiley & Sons, Inc., New York, 1999); Molecular Cloning: A Laboratory Manual, 2nd Ed., ed. by Sambrook, Fritsch, and Maniatis (Cold Spring Harbor Laboratory Press: 1989); Nucleic Acid Hybridization (B. D. Hames & S. J. Higgins eds. 1984)). The nucleic acid may be purified from whole cells using DNA or RNA purification techniques. The genetic sample may also be amplified using PCR or in vivo techniques requiring subcloning. The genetic sample can be obtained by isolating mRNA from the cells of the biological sample and reverse transcribing the RNA into DNA in order to create cDNA (Khan et al. Biochem. Biophys. Acta 1423:17 28, 1999).

Once a genetic sample has been obtained, it can be analyzed for the presence or absence of one or more particular genes encoding, for example, a steroid receptor coactivator such as SRC-3. The analysis may be performed using any techniques known in the art including, but not limited to, sequencing, PCR, RT-PCR, quantitative PCR, restriction fragment length polymorphism, hybridization techniques, Northern blot, microarray technology, DNA microarray technology, and the like. In determining the expression level of a gene or genes in a genetic sample, the level of expression may be normalized by comparison to the expression of another gene such as a well known, well characterized gene or a housekeeping gene. For example, reverse-transcriptase PCR (RT-PCR) can be used to detect the presence of a specific mRNA population in a complex mixture of thousands of other mRNA species. In RT-PCR, an mRNA species is first reverse transcribed to complementary DNA (cDNA) with use of the enzyme reverse transcriptase; the cDNA is then amplified as in a standard PCR reaction. RT-PCR can thus reveal by amplification the presence of a single species of mRNA.

Hybridization to clones or oligonucleotides arrayed on a solid support (i.e., gridding) can be used to both detect the expression of and quantitate the level of expression of that gene. In this approach, a cDNA encoding an antigen is fixed to a substrate. The substrate may be of any suitable type including but not limited to glass, nitrocellulose, nylon, or plastic. At least a portion of the DNA encoding the antigen is attached to the substrate and then incubated with the analyte, which may be RNA or a complementary DNA (cDNA) copy of the RNA, isolated from the sample of interest. Hybridization between the substrate bound DNA and the analyte can be detected and quantitated by several means including but not limited to radioactive labeling or fluorescence labeling of the analyte or a secondary molecule designed to detect the hybrid. Quantitation of the level of gene expression can be done by comparison of the intensity of the signal from the analyte compared with that determined from known standards. The standards can be obtained by in vitro transcription of the target gene, quantitating the yield, and then using that material to generate a standard curve.

V. Kits

Also provided herein are kits for treating or preventing cancer in a subject. A kit can include any of the compounds or compositions described herein. For example, a kit can include a compound of Formula I, Formula II, Formula III, or combinations thereof. A kit can further include one or more additional agents, such as one or more chemotherapeutic agents (e.g., gefitinib). A kit can include an oral formulation of any of the compounds or compositions described herein. A kit can include an intravenous formulation of any of the compounds or compositions described herein. A kit can additionally include directions for use of the kit (e.g., instructions for treating a subject), a container, a means for administering the compounds or compositions (e.g., a syringe), and/or a carrier.

As used herein the terms treatment, treat, or treating refer to a method of reducing one or more symptoms of a disease or condition. Thus in the disclosed method, treatment can refer to a 10%, 20%, 30%, 40%, 50%, 60%, 70%, 80%, 90%, or 100% reduction in the severity of one or more symptoms of the disease or condition. For example, a method for treating a disease is considered to be a treatment if there is a 10% reduction in one or more symptoms or signs (e.g., size of the tumor or rate of tumor growth) of the disease in a subject as compared to a control. As used herein, control refers to the untreated condition (e.g., the tumor cells not treated with the compounds and compositions described herein). Thus the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition.

As used herein, the terms prevent, preventing, and prevention of a disease or disorder refer to an action, for example, administration of a composition or therapeutic agent, that occurs before or at about the same time a subj ect begins to show one or more symptoms of the disease or disorder, which inhibits or delays onset or severity of one or more symptoms of the disease or disorder.

As used herein, references to decreasing, reducing, or inhibiting include a change of 10%, 20%, 30%, 40%, 50%, 60%, 70%, 80%, 90% or greater as compared to a control level. Such terms can include, but do not necessarily include, complete elimination.

As used herein, subject means both mammals and non-mammals. Mammals include, for example, humans; non-human primates, e.g., apes and monkeys; cattle; horses; sheep; rats; mice; pigs; and goats. Non-mammals include, for example, fish and birds.

Throughout this application, various publications are referenced. The disclosures of these publications in their entireties are hereby incorporated by reference into this application.

The examples below are intended to further illustrate certain aspects of the methods and compositions described herein, and are not intended to limit the scope of the claims.

### EXAMPLES

### Example 1

### Steroid Receptor Coactivator-3 (SRC-3/AIB1) as a Therapeutic Target in Triple Negative Breast Cancer and its Inhibition with a Phospho-Bufalin Prodrug

Triple negative breast cancer (TNBC) accounts for approximately 15-20% of breast cancer incidence. These tumors are highly aggressive, metastatic, and have the worst short-term prognosis among all breast cancer types. Unlike estrogen receptor positive (ER+) breast cancer, which can be effectively treated with aromatase inhibitors, tamoxifen or other SERMs, there is no targeted therapy currently approved for TNBC. Chemotherapy and surgery are the current standard-of-care (SOC) for TNBC. Besides the severe side effects associated with chemotherapies, the selection pressure induced by nonspecific chemotherapy drugs and the development of drug resistance can promote metastasis. Surgical removal of the primary tumor also may promote proliferation of metastases in part due to excessive release of growth factors intended for wound healing. Targeted therapy is limited because the vast majority of inhibitors can inhibit only one pathway. Consequently, drug resistance occurs when tumors use alternative growth signaling pathways, subverting the original therapeutic target. Thus, there is an urgent need to identify applicable targets and to develop novel treatments to improve TNBC treatment outcomes.

Bufalin directly binds to SRC-3 in its receptor interacting domain and selectively reduces the levels of SRC-3 in ER+breast cancer cell lines without perturbing overall protein expression patterns. Additionally, bufalin exhibits IC₅₀ values in the low nM range with selective toxicity towards cancer cells, keeping normal cell viability unperturbed. See Wang et al., Cancer Research, 74(5): 1506-1517 (2014), which is hereby incorporated by reference in its entirety. However, due to its low solubility, bufalin has limited use as a viable therapeutic agent. Provided herein is a prodrug strategy that converts water insoluble bufalin into a soluble analog: 3-phospho-bufalin (p-Buf). P-Buf can be hydrolyzed by endogenous phosphatases under physiological conditions to regenerate bufalin. This prodrug strategy avoids sudden exposure to high concentrations of free bufalin, which may cause acute cardiotoxicity. In addition, as demonstrated herein, p-Buf can significantly reduce tumor growth in an orthotopic TNBC model.

### Materials and Methods

Cell Culture. Triple negative breast cancer (TNBC) cell lines HCC1143, SUM149PT, SUM159PT, and MDA-MB-231 were obtained from ATCC. The MDA-MB-231-LM3-3 (LM3-3) cell line was developed from lung metastasis derived from a MDA-MB-231-LM2 xenograft tumor in the mammary fat pad of a SCID mouse.

The HCC1143 cell line was grown in RPMI-1640 medium, SUM149PT and SUM159PT cell lines were grown in F-12 medium, and MDA-MB-231 cells were grown in DMEM. All culture media were supplemented with 10% fetal bovine serum. All cell lines were grown in a humidified incubator containing 5% CO₂ at 37° C. Primary hepatocytes were isolated from an adult male mouse (C57BL) by collagenase perfusion (0.8 mg/mL; Sigma-Aldrich, C5138) through the portal vein and maintained in M199 medium with 10% FBS. The viability of freshly isolated hepatocytes was verified using Trypan blue staining.

Cell Cytotoxicity Assays. TNBC cells (5000 to 7000 cells per well) were seeded in 96-well plates in medium supplemented with 10% FBS. On the next day, when cells reached 70% to 80% confluence, bufalin (Santa Cruz) was added to achieve an array of final concentrations (0.2, 0.5, 1, 2, 5, 10, 20, 50, 100, 500 and 1000 nM). After 72 hours of bufalin treatment, cell viability was measured by MTT assay. Cell viabilities relative to untreated control cells were plotted using Graphpad Prism. The IC₅₀ values for bufalin were calculated based on the Hill-Slope model.

Western Blotting Assays. Cell lysate was prepared by adding RIPA buffer (containing 10 mM Tris-Cl pH 8.0, 1 mM EDTA, 0.5 mM EGTA, 1% Triton X-100, 0.1% sodium deoxycholate, 0.1% SDS, 140 mM NaCl, and protease inhibitors) to cells kept on ice. After spinning samples at 16,000 rpm for 15 minutes at 4 ° C., insoluble materials in the collected cell lysate were precipitated and discarded. The total protein concentration was measured using a Bradford protein assay. An equivalent mass of each sample was loaded and electrophoretically separated by 10% SDS-PAGE. Then, proteins on PAGE the gel were transferred to a PVDF membrane. Anti-SRC-3 (Cell Signaling Technology Co. Cat#2126) and anti-actin (Cell Signaling Technology Co. Cat#8457) were used to probe the membranes.

Single Cell Motility Assays. Microtiter 96-well plates pre-coated with collagen V were covered with beads provided by a Cell Motility HCS kit (Thermo Scientific Co. Cat# K0800011). LM3-3 cells (500) were seeded in each bead-coated well. After 18 hours of incubation, the cell tracks impressed upon the beads were fixed with 4% paraformaledhyde (PFA) and photographed with a phase contract microscope. The cell track areas (pixels) were counted using Image J. To attain statistical significance, more than 50 cell tracks in each sample were counted.

Synthesis of 3-Phospho-bufalin. 3-(Di-t-butyl-phospho)-bufalin: Bufalin (100 mg, 0.259 mmol) was dissolved in dichloromethane (DCM, 5 mL) under nitrogen. 1-H-tetrazole (3 mL, 0.45 M in CH₃CN) and di-t-butyl diethylphosphoramidite (0.15 mL, 0.518 mmol) were added in succession. The reaction was stirred at room temperature for 30 min and then cooled to −78° C. in a dry ice-acetone bath. m-Chloroperoxybenzoic acid (mCPBA, 134 mg, 0.777 mmol) in DCM (3 mL) was added dropwise. After 30 min, the reaction mixture was diluted with DCM (10 mL), washed sequentially with Na₂S₂O₃ solution, NaHCO₃ saturated solution, then brine, and dried over anhydrous sodium sulfate. The organic phase was filtered and concentrated under vacuum. The residue was purified by column chromatography on silica gel with hexanes/ethyl acetate eluent (1:2, v/v) to isolate 3-(di-t-butyl-phospho)-bufalin as a white solid (105 mg, two steps with a combined 72% yield). NMR analysis of this product:¹H NMR (400 MHz, CDCl₃): δ 7.83 (dd, J=9.6, 2.4 Hz, 1H), 7.22 (d, J=2.4 Hz, 1H), 6.25 (d, J=9.6 Hz, 1H), 4.66-4.68 (m, 1H), 2.44-2.48(m, 1H), 2.15-2.23(m, 1H), 2.00-2.08 (m, 1H), 1.33-1.88 (m, 13H), 1.48 (s, 18H), 1.21-1.40 (m, 7H), 0.94 (s, 3H), 0.70 (s, 3H).

3-Phospho-bufalin: 3-(Di-t-butyl-phospho)-bufalin (20 mg, 0.035 mmol) was dissolved in a mixture of DCM (14 mL) and methanol (2.8 mL), then added to 4N HCl in dioxane (1.0 mL) at 0° C. After stirring at 0° C. for 30 minutes, the solvent was removed under vacuum to afford the target product 3-phospho-bufalin as a white solid (14 mg, 85% yield). The purity of the product was determined to be >99% using high performance liquid chromatography (HPLC)). The product was used without further purification. NMR analysis of this product: ¹H NMR (400 MHz, CD₃OD): δ 7.99 (d, J=9.6 Hz, 1H), 7.43 (s, 1H), 6.28 (d, J=9.6 Hz, 1H), 4.65 (br, 1H), 2.53-2.56 (m, 1H), 2.11-2.21 (m, 2H), 1.93-2.03 (m, 2H),1.43-1.84 (m, 12 H), 1.21-1.29 (m, 6H), 0.98 (s, 3H), 0.72 (s, 3H); ESI-MS (m/z): [M+H]⁺ calculated for C₂₄H₃₆O₇P, 467.2; found, 467.0.

Electrocardiogram (EKG) Measurements. Female ICR mice (5 weeks old) were treated with bufalin or 3-phospho-bufalin via intravenous (i.v.) and intraperitoneal (i.p.) injections, respectively, at a range of concentrations. Non-invasive EKG recordings were made using the ECGenie system (Mouse Specifics) at several subsequent time points. All data were collected at a similar hour in the day, as the heart rate is most stable during “inactive” daylight hours. Data acquisition was performed using the program LabChart 6 (ADlnstruments). Individual EKG signals were then evaluated using e-MOUSE physiologic waveform analysis software (Mouse Specifics) as previously described.

Pharmacokinetic Studies of Bufalin and 3-phospho-bufalin. Bufalin (0.5 mg/kg, i.v.) or 3-phospho-bufalin (0.5 mg/kg, i.p.) were injected into IRC mice (n=3). Twenty microliters of blood was collected via the tail vein at 5, 15, 30 min, and 1, 2, 4, 6, 12, and 24 h.

To calibrate the quantification of bufalin and 3-phospho-bufalin, solutions of each analyte were prepared by serial dilution in 50% acetonitrile in water. Five quality control (QC) samples at 10 ng/mL, 20 ng/mL, 500 ng/mL, 4000 ng/mL and 8000 ng/mL of plasma were prepared independently of those used for the calibration curves. QC samples were prepared on the day of analysis in the same way as the calibration standards.

Standards, QC samples, and unknown samples were injected into a LC/MS/MS machine for quantitative analysis. The pharmacokinetic parameters of bufalin and P-Buf were analyzed using WinNonLin. For i.v. injected bufalin, the PK trace was fitted into a non-compartmental i.v. bolus model. For i.p. injected P-Buf, the PK trace was fittedto a non-compartmental extravascular model.

Therapeutic Efficacy of 3-phospho-bufalin in an Orthotopic TNBC Mouse Model. To establish orthotopic breast tumors, 0.75×10⁶ MDA-MB-231-LM3-3 cells were injected into one of the second mammary fat pads of nude mice (Charles River Laboratories, female, 6-7 weeks, n=6/group). When tumors became palpable, generally 14 days after injection, mice were randomized into two groups. The treatment group was then injected with phospho-bufalin subcutaneously (0.75 mg/kg per dose, 3 doses per week) for 3 weeks while the control group received PBS. Tumor lengths and widths were measured three times per week. Tumor sizes were calculated by: (length×width×width)/2.

### Results

Overexpression of SRC-3 Correlates with Poor Prognosis in TNBC Patients

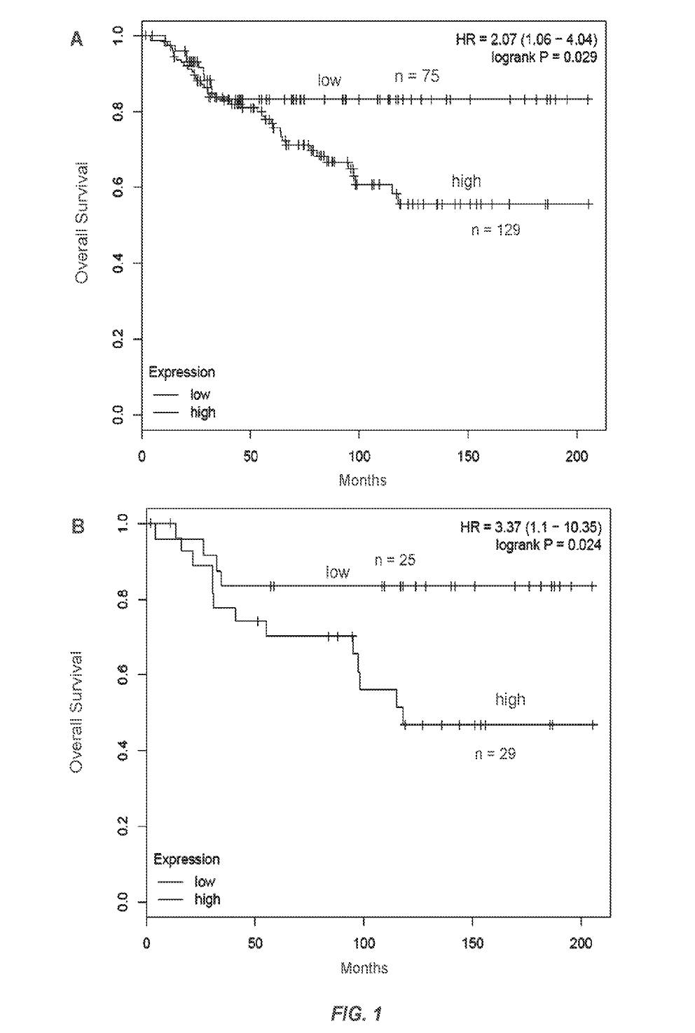

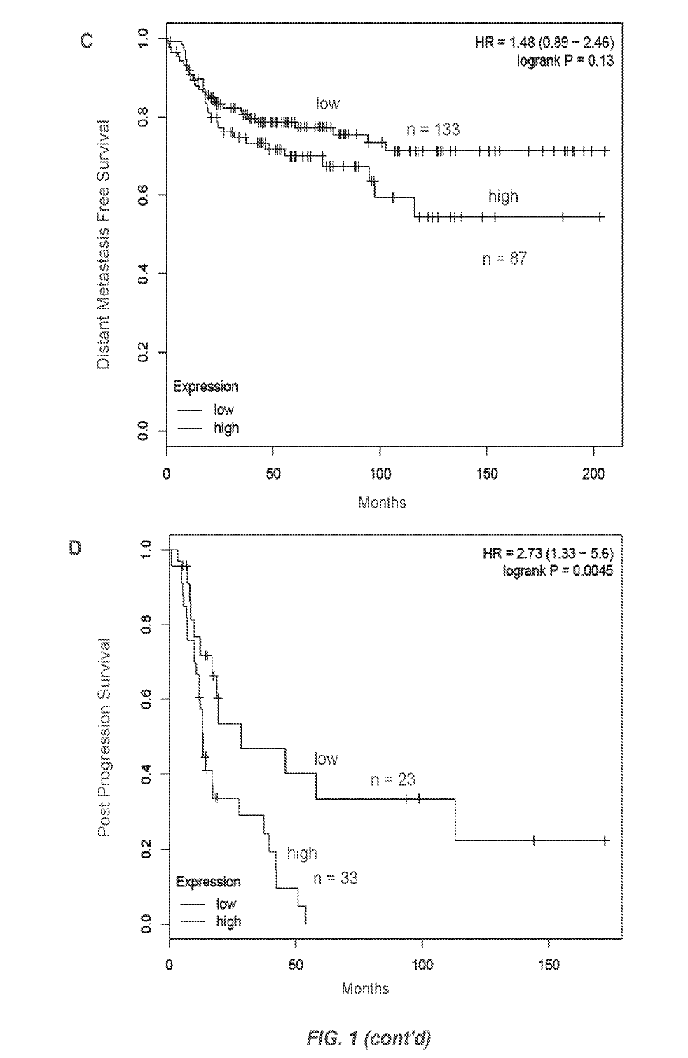

The KM-plotter microarray data from breast cancer patients comprising basal-like intrinsic subtypes were analyzed, 80-100% of which are generally triple negative. It was found that high expression of SRC-3 is inversely correlated with overall survival (OS, Log Rank P=0.029, HR=2.07, FIG. 1A). A similar trend was observed for the overall survival of systemically untreated TNBC patients (Log Rank P=0.024, HR=3.37, FIG. 1B). Overexpression of SRC-3 also correlates with poor distant-metastasis-free survival (DMFS, Log Rank P=0.13, HR=1.48, FIG. 1C. In addition, post-progression survival is markedly lower in patients with high levels of SRC-3 (PPS, Log Rank P=0.0045, HR=2.73, FIG. 1D). Overall, overexpression of SRC-3 correlates with poor prognosis in TNBC patients.

Inhibition of SRC-3 by Bufalin Potently Reduces TNBC Cell Viability, but Spares Primary Hepatocytes

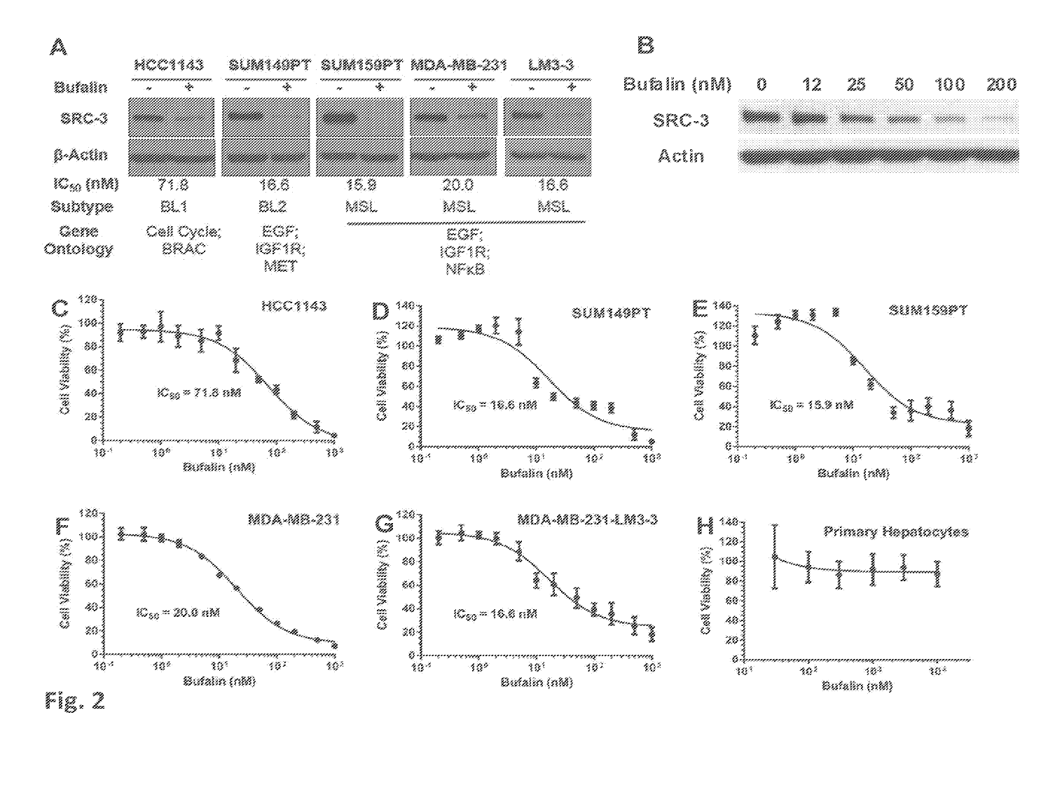

Bufalin is a potent small molecule inhibitor of SRC-3. As shown in FIG. 2A, Western blotting results show that 100 nM of bufalin can downregulate SRC-3 protein expression in a panel of TNBC cell lines- HCC1143, SUM149PT, SUM159PT, MDA-MB-231, and MDA-MB-231-LM3-3. In addition, bufalin downregulated SRC-3 in MDA-MB-231-LM3-3 in a dose-dependent manner (FIG. 2B).

Low nM concentrations of bufalin can significantly reduce the viability of ER+ breast cancer cells. TNBC cell lines were treated with a range of bufalin concentrations for 72 h. Cell viabilities were measured and compared against untreated control cells by MTT assay. It was found that the growth of all TNBC cell lines was potently inhibited by bufalin with IC₅₀ values lower than 20 nM (FIG. 2D-G), with the single exception in HCC1143 where an IC₅₀=71.8 nM was observed (FIG. 2C).

Bufalin is mainly excreted through hepatocyte clearance via a biliary route. Therefore, bufalin's liver toxicity should be considered during the drug development process.

Remarkably, even at concentrations of up to 300 nM, the highest concentration treatment, bufalin did not cause any observable toxicity in mouse primary hepatocytes (FIG. 2H). This selective cytotoxicity bodes well for bufalin's candidacy as a potential anticancer drug.

Inhibition of SRC-3 by Bufalin Decreases TNBC Cell Motility

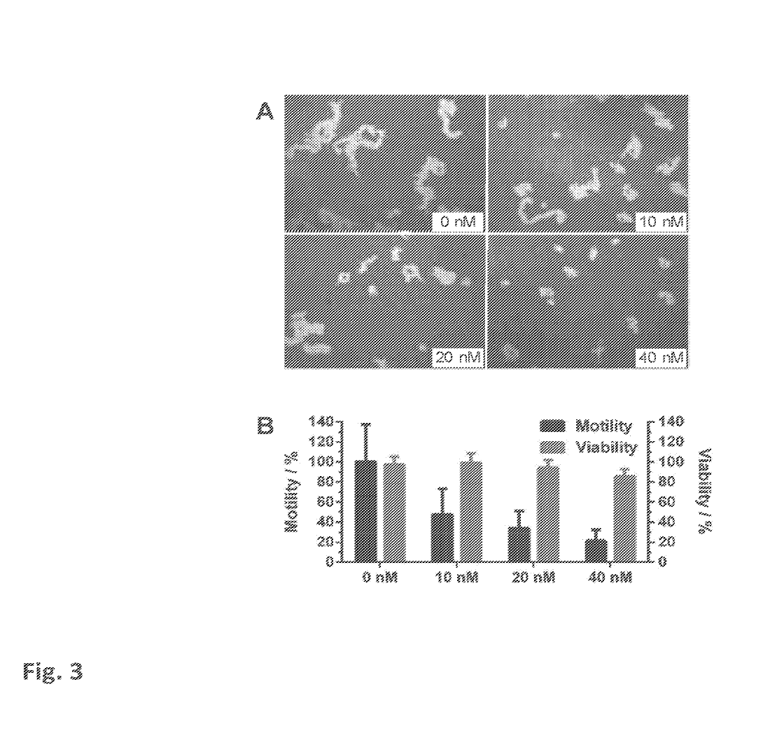

Down-regulation of SRC-3 can inhibit cell motility, invasion, and tumor metastasis. The single cell motility assay showed that the motility of LM3-3 cells decreased with increasing concentrations of bufalin (FIGS. 3A and 3B). The experimental conditions used in this assay produced minimal toxicity toward LM3-3 cells at the 12 hour time point (FIG. 3B), indicating that the observed motility difference was not due to decreased viability.

Bufalin Synergizes with Gefitinib in Triple Negative Breast Cancer Cells

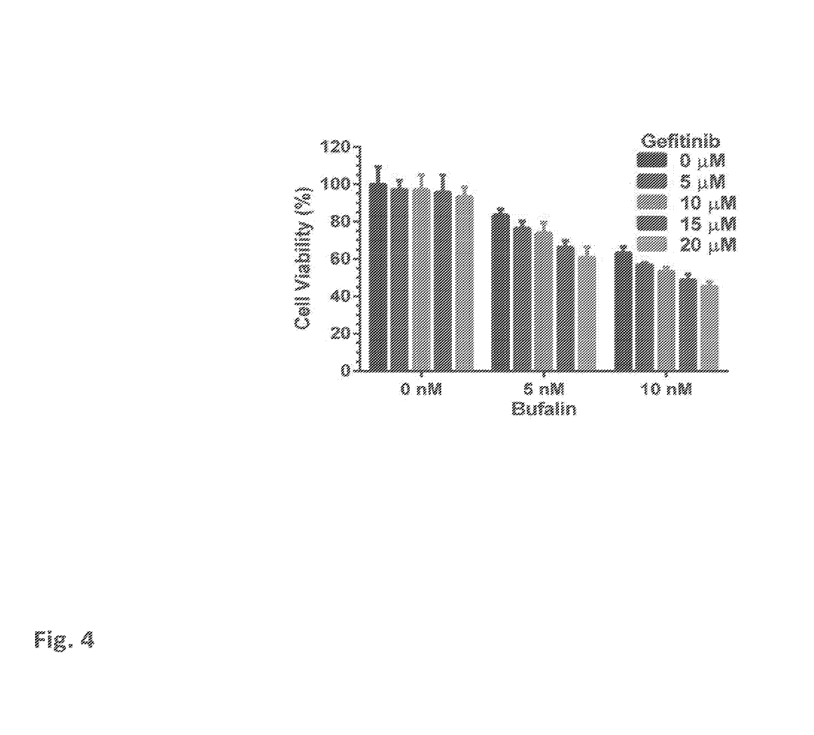

Despite the popularity of tyrosine kinase inhibitors (TKIs) in conventional cancer therapies, none have been found to be successful for the treatment of TNBC. However, over-expression of EGFR is a hallmark of TNBC and usually correlates with poor prognosis. The inability of EGFR inhibitors, such as gefitinib, to arrest TNBC growth has been attributed to crosstalk between growth factors. Despite the fact that gefitinib, at concentrations up to 20 μM caused minimal toxicity in LM3-3 cells, bufalin can synergize with gefitinib in TNBC cells, likely through crosstalk inhibition, given SRC-3's role as a growth factor signaling integrator (FIG. 4).

Synthesis of 3-Phospho-bufalin

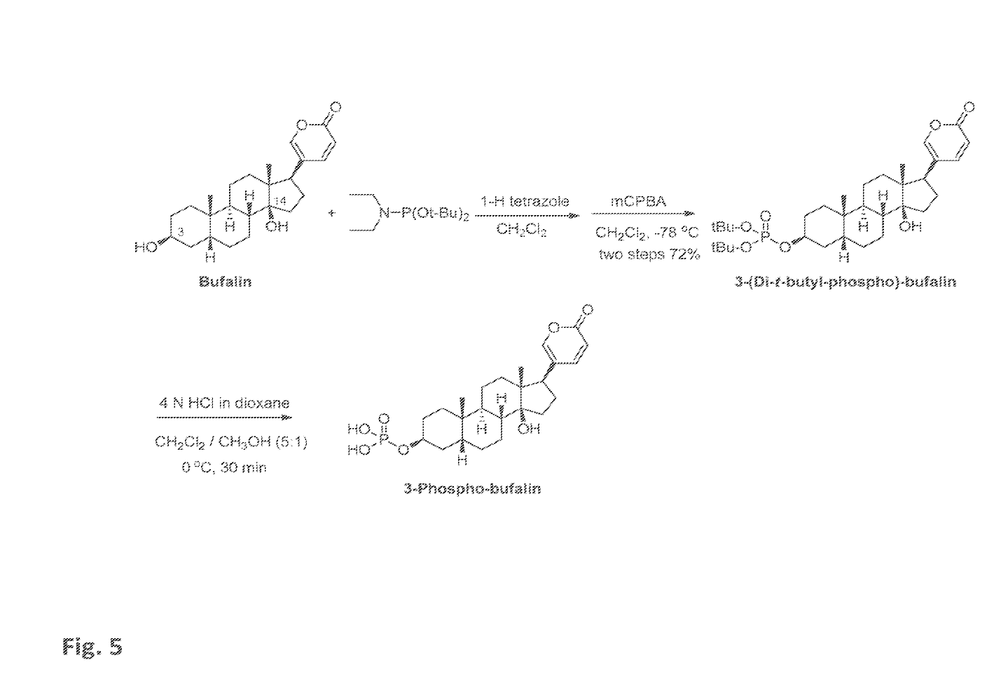

As shown in FIG. 5, bufalin was converted to 3-(di-t-butyl-phospho)-bufalin through reaction with di-t-butyl diethylphosphoramidite and subsequent oxidation with m-chloroperoxybenzoic acid (mCPBA). The t-butyl groups were carefully de-protected in an acidic environment to supply the final P-Buf. The phosphate group now can be cleaved by endogenous phosphatases to regenerate free bufalin.

3-Phospho-bufalin Reduces Cardiotoxicity in vivo

Because cardiac glycosides can cause cardiotoxicity, their clinical application necessitates close monitoring. Despite the fact that there are many in vitro assays available to predict cardiotoxicity of a drug candidate, in vivo electrocardiogram measurements remain the gold standard for this assessment.

Intravenous injection of free bufalin (0.5 mg/kg) caused significant decreases in heart rate and an increase in EKG parameters five min post-injection (Table 1). The changes in heart rate and electrophysiology are similar to the observed cardiotoxicity caused by digoxin. An observed QTc interval prolongation may indicate altered K⁺ or Ca²⁺ ion channel function during repolarization. The cardiotoxicity caused by bufalin (0.5 mg/kg) appears to be transient and most of the electrophysiology data reflected a return to normal cardiac activity 24 h post-injection. Additionally, the injection dose of free bufalin was decreased from 0.5 to 0.1 mg/kg and no acute cardiotoxicity was found.

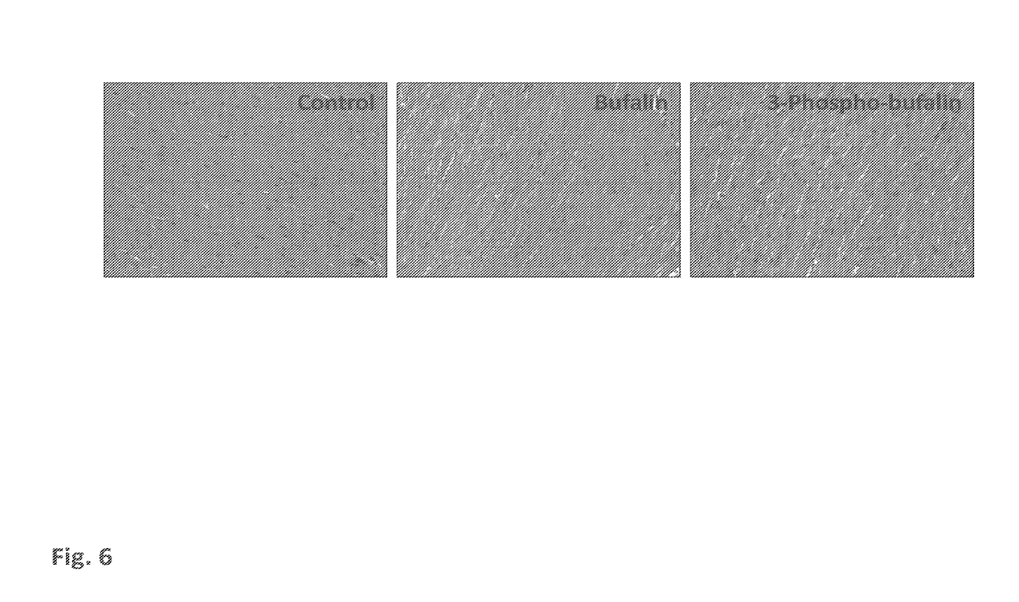

In contrast, following 3-phospho-bufalin administration even at 7.5 mg/kg and 11.25 mg/kg, no significant changes in EKG parameters were observed. Therefore, 3-phospho-bufalin has no acute cardiotoxicity at these higher doses, demonstrating it may be a safer agent than its parent drug, bufalin. In addition, histological examinations found little damage to cardiomyocytes treated with bufalin and P-Buf (FIG. 6).

TABLE 1

| | Dose | Post- | | HR | RR | QRS | QT | QTc | ST |
|---|---|---|---|---|---|---|---|---|---|
| | (mg/kg) | injection | | (BPM) | (ms) | (ms) | (ms) | (ms) | (ms) |
| Untreated | 0 | −5 | min | 799.0 ± 3.0 | 75.1 ± 0.3 | 10.4 ± 0.2 | 37.8 ± 2.1 | 43.6 ± 2.4 | 27.9 ± 2.0 |
| | | +10 | min | 805.5 ± 17.7 | 77.1 ± 5.3 | 10.1 ± 0.5 | 39.0 ± 2.2 | 45.0 ± 1.6 | 29.5 ± 2.6 |
| Bufalin | 0.1 | −5 | min | 746 ± 15.8 | 82.2 ± 6.9 | 10.2 ± 0.4 | 40.2 ± 1.8 | 44.7 ± 1.1 | 30.6 ± 1.9 |
| | | +5 | min | 724 ± 5.7 | 88.5 ± 0.4 | 10.0 ± 0.1 | 41.0 ± 0.7 | 44.2 ± 0.6 | 31.6 ± 0.8 |
| | 0.2 | −5 | min | 769.3 ± 17.2 | 78.0 ± 1.8 | 9.2 ± 0.1 | 41.2 ± 1.8 | 46.7 ± 1.8 | 32.5 ± 1.7 |
| | | +5 | min | 500.3 ± 24.5 | 123.6 ± 9.5 | 10.9 ± 0.6 | 55.6 ± 10.2 | 49.8 ± 4.8 | 45.2 ± 9.6 |
| | 0.3 | −5 | min | 683.3 ± 18.7 | 89.4 ± 0.6 | 10.4 ± 1.6 | 46.1 ± 0.5 | 48.3 ± 1.3 | 36.3 ± 1.9 |
| | | +5 | min | 464.7 ± 5.7 | 131.0 ± 0.9 | 14.4 ± 0.2 | 61.4 ± 1.6 | 53.7 ± 1.3 | 47.5 ± 1.3 |
| | 0.5 | −5 | min | 761.1 ± 10.9 | 78.9 ± 1.1 | 11.1 ± 0.2 | 39.9 ± 1.5 | 44.9 ± 1.8 | 29.3 ± 1.5 |
| | | +5 | min | 582.2 ± 30.5 | 103.7 ± 5.3 | 13.4 ± 0.3 | 51.4 ± 3.0 | 50.4 ± 1.6 | 38.5 ± 3.3 |
| | | +24 | h | 766.1 ± 2.5 | 78.4 ± 0.3 | 10.7 ± 0.2 | 41.9 ± 1.1 | 47.3 ± 1.2 | 28.7 ± 1.3 |
| 3- | 7.5 | −5 | min | 754.7 ± 6.6 | 79.6 ± 0.7 | 10.6 ± 0.1 | 40.2 ± 0.6 | 45.0 ± 0.5 | 30.1 ± 0.7 |
| Phospho- | | +10 | min | 788.7 ± 1.2 | 76.1 ± 0.1 | 11.0 ± 0.1 | 38.2 ± 0.9 | 43.8 ± 1.0 | 27.6 ± 0.9 |
| Bufalin | 11.25 | −5 | min | 798.0 ± 12.8 | 75.8 ± 0.2 | 10.6 ± 0.3 | 39.1 ± 2.4 | 45.1 ± 2.9 | 29.0 ± 2.2 |
| | | +5 | min | 779.7 ± 1.5 | 77.0 ± 0.1 | 10.9 ± 0.1 | 37.1 ± 0.3 | 42.3 ± 0.4 | 26.7 ± 0.4 |
| | | +10 | min | 761.3 ± 7.5 | 78.8 ± 0.8 | 11.1 ± 0.2 | 38.1 ± 0.6 | 43.0 ± 0.6 | 26.6 ± 0.5 |
| | | +15 | min | 755.0 ± 1.0 | 79.5 ± 0.1 | 11.5 ± 0.1 | 39.1 ± 0.9 | 43.9 ± 0.9 | 28.1 ± 0.8 |

Pharmacokinetics of Bufalin and 3-Phospho-bufalin

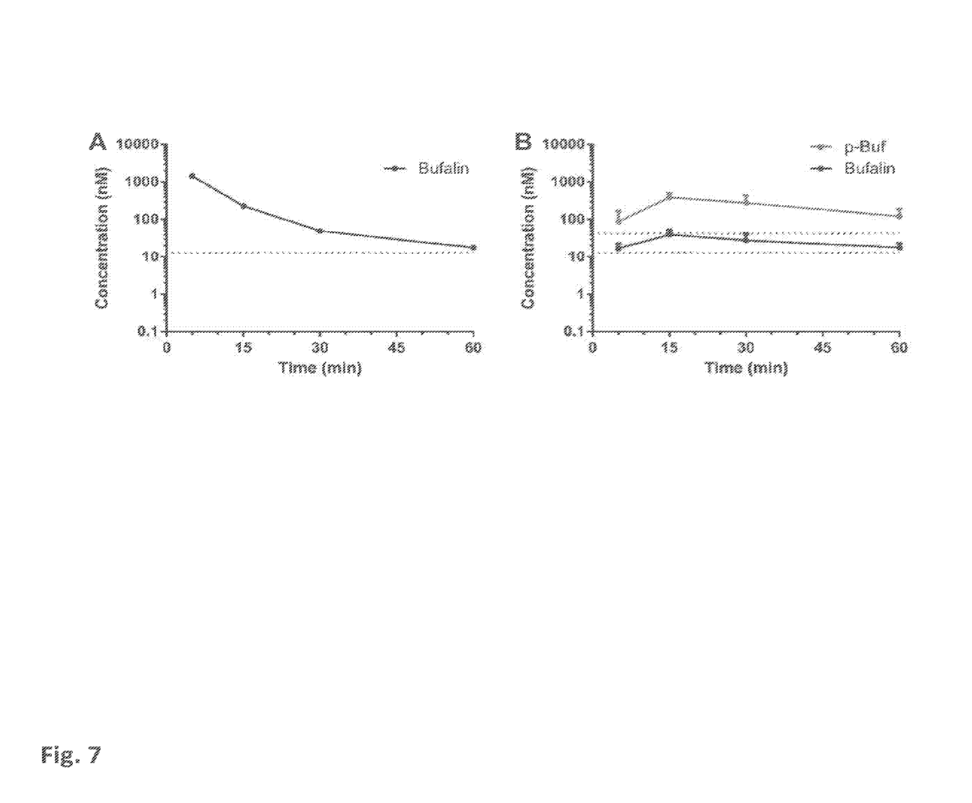

The pharmacokinetic profiles of bufalin and P-Buf were measured in ICR mice. Bufalin was dissolved in a mixture of 1,2-propanediol and PBS (v/v,75/25) and 0.5 mg/kg administered intravenously. P-Buf was dissolved in PBS and administered intraperitoneally at the same dose. Blood (20 μL for each time point) was collected at 5, 15, 30 min, and 1, 2, 4, 6, 12, and 24 h. The plasma concentrations of bufalin and p-buf were analyzed using LC/MS/MS. The pharmacokinetic traces for bufalin and p-buf are shown in FIG. 7.

Bufalin exhibited a short half-life of 0.177±0.078 h in treated mice (FIG. 7A). One hour post administration, the plasma concentration of bufalin decreased to the lowest limit of quantification (LLOQ, blue dash line in FIG. 7A). The extrapolated plasma concentration of bufalin was at time zero (C₀) is 3.62±0.71 μM. However, for the second treatment group (i.p., FIG. 7B), both p-Buf and free bufalin were detected in plasma during the first hour. Bufalin half-life is extended 4-fold compared to those of the first group, possibly due to the absorption and hydrolysis of p-buf. Detection of free bufalin in plasma also confirms that p-buf can be converted to parent bufalin in vivo. All the pharmacokinetic parameters are summarized in Table 2.

TABLE 2

| | | Dose | Cl_obs | t_{1/2} | tₘₐₓ | C₀ | AUCₗₐₛₜ | AUC_{Inf} | AUCₗₐₛₜ/D | Vₛₛ_{_{—}}_{obs} |
|---|---|---|---|---|---|---|---|---|---|---|
| Treatment | Route | (mg/kg) | (mL/min/kg) | (h) | (h) | (μM) | (μM * h) | (μM * h) | (μM * h * kg/mg) | (L/kg) |
| bufalin | i.v. | 0.5 | 54.3 ± 8.4 | 0.177 ± 0.078 | NA | 3.62 ± 0.71 | 0.396 ± 0.058 | 0.402 ± 0.058 | 0.791 ± 0.116 | 0.368 ± 0.138 |
| p-bufain | i.p. | 0.5 | NA | 0.742 ± 0.104 | 0.25 | NA | 0.031 ± 0.018 | 0.046 ± 0.018 | 0.075 ± 0.044 | NA |

3-Phospho-bufalin Inhibits Tumor Growth in an Orthotopic TNBC model

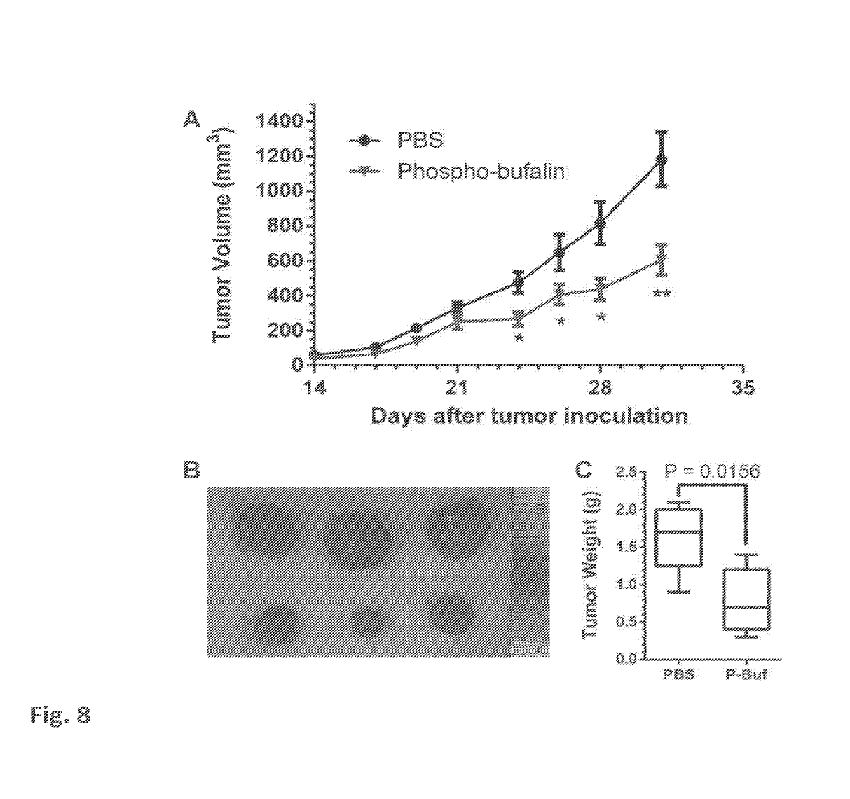

The therapeutic efficacy of P-Buf was tested in an orthotopic TNBC model. LM3-3 cells (0.75×10⁶ cells per mouse) were inoculated into the second pair of mammary gland fat pads of nude mice (female, 4-5 weeks). The mice were randomized into two groups based on luciferase imaging (n=6 per group). Fourteen days after tumor inoculation, the treatment group began receiving P-Buf (0.75 mg/kg) three times per week while the control group received PBS. Tumor volumes were measured three times per week. As shown in FIG. 8A, P-Buf can significantly inhibit TNBC tumor growth. At day 31, the mice were euthanized and tumors were harvested. The tumors derived from PBS treated mice weighed ˜2.4 times more than those of the experimental treatment group (FIGS. 8B and C).

The compounds and methods of the appended claims are not limited in scope by the specific compounds and methods described herein, which are intended as illustrations of a few aspects of the claims and any compounds and methods that are functionally equivalent are within the scope of this disclosure. Various modifications of the compounds and methods in addition to those shown and described herein are intended to fall within the scope of the appended claims. Further, while only certain representative compounds, methods, and aspects of these compounds and methods are specifically described, other compounds and methods are intended to fall within the scope of the appended claims. Thus, a combination of steps, elements, components, or constituents can be explicitly mentioned herein; however, all other combinations of steps, elements, components, and constituents are included, even though not explicitly stated.

1. A compound of the following formula: 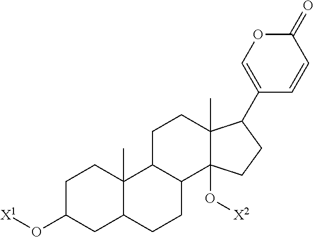
or a pharmaceutically acceptable salt thereof, wherein:
X¹ and X² are each independently selected from the group consisting of hydrogen and 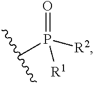
wherein R¹ and R² are each independently selected from the group consisting of hydroxy, substituted or unsubstituted alkoxy, substituted or unsubstituted alkenyloxy, substituted or unsubstituted alkynyloxy, substituted or unsubstituted heteroalkyloxy, substituted or unsubstituted heteroalkenyloxy, substituted or unsubstituted heteroalkynyloxy, substituted or unsubstituted aryloxy, substituted or unsubstituted heteroaryloxy, substituted or unsubstituted cycloalkyloxy, substituted or unsubstituted heterocycloalkyloxy, and substituted or unsubstituted amino; and
wherein X¹ and X² are not simultaneously hydrogen.

2. The compound of claim 1, wherein the compound has the following formula: 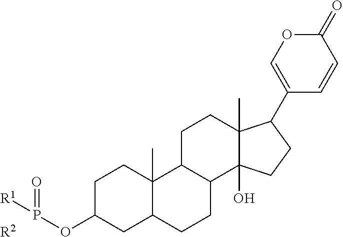
or a pharmaceutically acceptable salt or prodrug thereof.

3. The compound of claim 1, wherein the compound has the following formula: 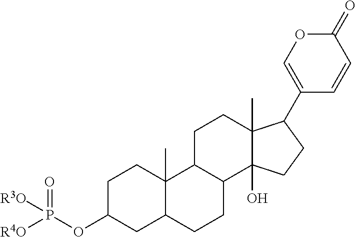
or a pharmaceutically acceptable salt thereof, wherein:
R³ and R⁴ are each independently selected from the group consisting of hydrogen, substituted or unsubstituted alkyl, substituted or unsubstituted alkenyl, substituted or unsubstituted alkynyl, substituted or unsubstituted heteroalkyl, substituted or unsubstituted heteroalkenyl, substituted or unsubstituted heteroalkynyl, substituted or unsubstituted aryl, substituted or unsubstituted heteroaryl, substituted or unsubstituted cycloalkyl, and substituted or unsubstituted heterocycloalkyl.

4. The compound of claim 1, wherein the compound has the following formula: 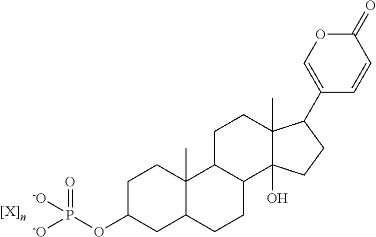
or a pharmaceutically acceptable salt thereof, wherein:
X is a cation; and
n is 1 or 2.

5. The compound of claim 4, wherein the cation is a metal cation.

6. The compound of claim 5, wherein the metal cation is an alkali metal cation or an alkaline earth metal cation.

7. The compound of claim 4, wherein X is selected from the group consisting of Na⁺, K⁺, Li⁺, and NH₄⁺.

8. The compound of claim 1, wherein the compound has the following formula: 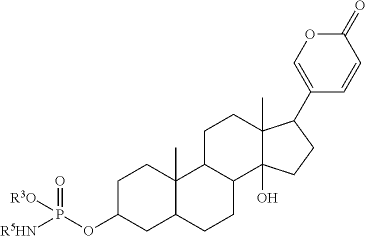
or a pharmaceutically acceptable salt thereof, wherein:
R³ and R⁵ are each independently selected from the group consisting of hydrogen, substituted or unsubstituted alkyl, substituted or unsubstituted alkenyl, substituted or unsubstituted alkynyl, substituted or unsubstituted heteroalkyl, substituted or unsubstituted heteroalkenyl, substituted or unsubstituted heteroalkynyl, substituted or unsubstituted aryl, substituted or unsubstituted heteroaryl, substituted or unsubstituted cycloalkyl, and substituted or unsubstituted heterocycloalkyl.

9. The compound of claim 1, wherein the compound is

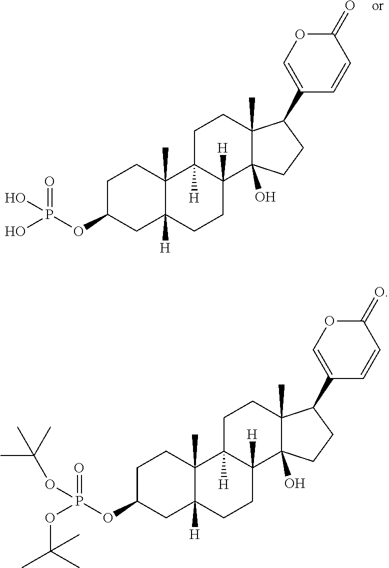

10. A composition comprising a compound of claim 1 and a pharmaceutically acceptable carrier.

11. (canceled)

12. A method of treating or preventing a steroid receptor coactivator-related disease in a subject, comprising:
administering to the subject an effective amount of a compound of claim 1.

13. The method of claim 12, wherein the steroid receptor coactivator-related disease is cancer, obesity, or human immunodeficiency virus.

14. The method of claim 13, wherein the steroid receptor coactivator-related disease is cancer and the cancer is a poor prognosis or invasive cancer, breast cancer, pancreatic cancer, glioblastoma, liver cancer, lung cancer, pancreatic cancer, or prostate cancer.

15. (canceled)

16. The method of claim 14, wherein the cancer is breast cancer and the breast cancer is triple negative breast cancer.

17. (canceled)

18. (canceled)

19. The method of claim 14, wherein the cancer is glioblastoma and the glioblastoma is a glioblastoma multiforme tumor.

20. The method of claim 19, wherein the glioblastoma multiforme tumor is a pediatric glioblastoma multiforme tumor.

21. (canceled)

22. (canceled)

23. (canceled)

24. (canceled)

25. The method of claim 12, further comprising administering a second compound or composition.

26. The method of claim 25, wherein the second compound or composition is a chemotherapeutic agent.

27. The method of claim 26, wherein the chemotherapeutic agent is gefitinib.

28. A method of inhibiting a steroid receptor coactivator protein in a cell, comprising:
contacting a cell with an effective amount of a compound of claim 1.

29. The method of claim 28, wherein the steroid receptor coactivator protein is SRC-1, SRC-2, or SRC-3.

30. (canceled)

31. (canceled)

32. The method of claim 28, wherein the contacting is performed in vitro or in vivo.

33. (canceled)

34. A method of identifying a subject at risk for developing a poor prognosis cancer, comprising:
(a) obtaining a biological sample from a subject; and
(b) detecting the expression of SRC-3 in the subject, wherein an increase in expression of SRC-3 in the subject as compared to SRC-3 in a control subject is indicative of a subject at risk for developing a poor prognosis cancer.

35. The method of claim 34, wherein the poor prognosis cancer is triple negative breast cancer.

36. A method of treating a subject at risk for developing a poor prognosis cancer, comprising:
(a) identifying a subject at risk for developing a poor prognosis cancer, comprising obtaining a biological sample from a subject and detecting the expression of SRC-3 in the subject, wherein an increase in expression of SRC-3 in the subject as compared to SRC-3 in a control subject is indicative of a subject at risk for developing a poor prognosis cancer; and
(b) administering to the subject an effective amount of a compound of claim 1.
